(19) United States
(12) Patent Application Publication
(10) Pub. No.: US 2015/0094518 A1
(43) Pub. Date: Apr. 2, 2015

(54) MODULAR POLYMER PLATFORM FOR THE TREATMENT OF CANCER

(71) Applicant: The Regents of the University of California, Los Angeles, CA (US)

(72) Inventors: Benjamin M. Wu, San Marino, CA (US); Maie A.R. St. John, Los Angeles, CA (US); Arnold Suwarnasam, Los Angeles, CA (US); Linda Wang, Irvine, CA (US); Yuan Lin, Los Angeles, CA (US); Jie Luo, Redondo Beach, CA (US); Eric Zhu, Los Angeles, CA (US); Ontario D. Lau, Los Angeles, CA (US)

(21) Appl. No.: 14/498,005

(22) Filed: Sep. 26, 2014

Related U.S. Application Data
(60) Provisional application No. 61/883,521, filed on Sep. 27, 2013.

(51) Int. Cl. A61L 31/16; A61N 5/10; A61L 31/06; G01N 33/543; A61K 41/00; A61L 31/14

(52) U.S. Cl. CPC A61L 31/16; A61K 41/0057; A61L 31/145; A61L 31/06; G01N 33/54306; A61N 5/10; A61L 2300/426; A61L 2300/224; G01N 2800/52; A61N 2005/1098
USPC 600/1; 424/85.1; 424/423

(57) ABSTRACT

The invention provides a novel polymer platform to deliver a desired combination of therapeutic agents to a site in need thereof for the treatment of cancer. In certain embodiments the platform is a modular polymer platform that allows for customization based upon the tumor of the subject to be treated.

### CROSS-REFERENCE TO RELATED APPLICATIONS

This application claims priority to U.S. Provisional Patent Application No. 61/883,521 filed Sep. 27, 2013, the contents of which are incorporated by reference herein in their entirety.

### STATEMENT REGARDING FEDERALLY SPONSORED RESEARCH OR DEVELOPMENT

This invention was made with Government support under DE021193, awarded by the National Institutes of Health. The Government has certain rights in the invention.

### BACKGROUND OF THE INVENTION

Head & Neck Squamous Cell carcinoma (HNSCC) is the sixth most common cancer in the world. Patients with HNSCC are at considerable risk of mortality, with more than 300,000 deaths attributable to the disease annually (Ferlay et al., GLOBOCAN 2000: Cancer incidence, mortality and prevalence worldwide, version 1.0. IARC Cancer Base No. 5). Aggressive surgical resection, with or without adjuvant chemoradiation (CRT) is the cornerstone of treatment for early disease. In many patients, the necessary surgery can be disfiguring and may also affect every day functioning, with a profound impact on quality of life (Shikani and Domb, 2000, Laryngoscope, 907-917). During the past 30 years, the 3- to 5-year survival rate of patients with advanced T3 and T4 HNSCC has remained poor (20-30%) despite considerable advances in surgical techniques and irradiation delivery and improvement in chemotherapeutic strategies. Because 50% of the patients with advanced and unresectable disease fail primary management, salvage in these patients is of paramount importance (Ross et al., 2004, Laryngoscope, 114: 1170-1176). Many of these patients receive radiation (RT) as definitive or as adjuvant therapy, which makes retreatment a challenge. Currently, the standard of care for recurrent disease is surgical salvage. Unfortunately, many advanced head and neck cancers are unresectable due to their proximity to vital structures such as the carotid artery or the skull base. Although palliation by chemotherapy is often attempted, systemic toxicity and its impact on the quality of life of patients prevents its wider clinical application (Xian et al., 2004, Arch Otolaryngol Head Neck Surg, 131: 1079-1085).

Therefore, there is a need in the art for compositions and methods for the treatment of cancer, including HNSCC. The present invention satisfies this unmet need.

### SUMMARY OF THE INVENTION

The present invention provides a modular polymer platform for the controlled delivery of two or more therapeutic agents. The platform comprises a polymeric substrate comprising two or more releasable therapeutic agents.

In one embodiment, at least one of the two or more therapeutic agents is an anti-tumor agent. In one embodiment, the anti-tumor agent is a chemotherapeutic agent. In one embodiment, at least one of the two or more therapeutic agents is an immunomodulator. In certain embodiments, the immunomodulator is at least one of CCL21, IL-2, IL-6, IL-8, IL-7, IL-10, IL-12, interferon, G-CSF, imiquimod, CCL3, CCL26, CXCL7, oligodeoxynucleotides, and glucan. In one embodiment, the substrate comprises a cell genetically modified to express the immunomodulator.

In one embodiment, the polymeric substrate comprises two or more layers, and wherein each layer has associated therewith at least one therapeutic agent. In one embodiment, a first layer of the substrate is a hydrogel. In one embodiment, a second layer is a polymer matrix. In one embodiment, the polymer matrix comprises PCL and PLCL. In one embodiment, the first layer and second layer release the at least one therapeutic agent comprised therein at different rates.

In one embodiment, polymeric substrate comprises an impermeable backing layer. In one embodiment, at least one of the two or more therapeutic agents is a radiosensitzer. In one embodiment, at least one of the two or more therapeutic agents is a radioprotective agent.

In one embodiment, the present invention provides a modular polymer platform for controlled delivery of one or more therapeutic agents, where the platform comprises a polymeric substrate comprising a cell genetically modified to express and secrete at least one of the one or more therapeutic agents. In one embodiment, at least one of the one or more therapeutic agents is an immunomodulator. In certain embodiments, the immunomodulator is at least one of CCL21, IL-2, IL-6, IL-8, IL-7, IL-10, IL-12, interferon, G-CSF, imiquimod, CCL3, CCL26, CXCL7, oligodeoxynucleotides, and glucan. In one embodiment, at least one of the one or more therapeutic agents is a chemotherapeutic agent. In one embodiment, the platform comprises a hydrogel layer comprising the cell.

In one embodiment, the present invention provides a modular polymer platform for controlled delivery of one or more therapeutic agents, where the platform comprises a polymeric substrate comprising recombinant CCL21. In one embodiment, at least one of the one or more therapeutic agents is a chemotherapeutic agent.

The present invention provides a method of treating or preventing cancer in a subject. The method comprises contacting tissue at or near the site of a tumor in a subject with a polymeric substrate that releases two or more therapeutic agents, wherein at least one of the two or more therapeutic agents is an anti-tumor agent. In one embodiment, the anti-tumor agent is a chemotherapeutic agent. In one embodiment, at least one of the two or more therapeutic agents is an immunomodulator In certain embodiments, the immunomodulator is at least one of CCL21, IL-2, IL-6, IL-8, IL-7, IL-10, IL-12, interferon, G-CSF, imiquimod, CCL3, CCL26, CXCL7, oligodeoxynucleotides, and glucan. In one embodiment, the substrate comprises a cell genetically modified to express the immunomodulator.

In one embodiment, the polymeric substrate comprises two or more layers, and wherein each layer has associated therewith at least one therapeutic agent. In one embodiment, a first layer of the substrate is a hydrogel. In one embodiment, a second layer of the substrate is a polymer matrix. In one embodiment, the polymer matrix comprises PCL and PLCL. In one embodiment, the method comprises releasing the at least one therapeutic agent comprised within the first and second layer at different rates.

In one embodiment, polymeric substrate comprises an impermeable backing layer. In one embodiment, at least one of the two or more therapeutic agents is a radiosensitizer. In one embodiment, at least one of the two or more therapeutic agents is a radioprotective agent.

In one embodiment, the cancer treated and prevented by the method is head and neck squamous cell carcinoma (HNSCC).

In one embodiment, the substrate is administered to the subject during surgical resection of at least part of the tumor. In one embodiment, the method comprises administering a low dose of radiation therapy to the subject.

In one embodiment, the method provides a personalized therapy by profiling the cancer of a subject and designing the polymer platform based upon the profiling of the cancer. In one embodiment, the profiling comprises obtaining a sample of the cancer and determining the drug sensitivity of the cancer. In one embodiment, the profiling comprises obtaining a biomarker profile of the subject. In one embodiment, the design of the polymer platform comprises determining at least one of the identities, concentration, and release characteristics, of each of the two or more therapeutic agents of the platform.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a graph demonstrating that cisplatin polymer effectively reduces the growth of mouse SCCVII/SF. 4×10⁵ SCVII/SF tumor cells were inoculated in C3H mice. The implanted cisplatin polymer significantly reduced tumor growth by 16 fold compared to controls. n=8 mice/group,** p<0.01 compared to surgery only.

FIG. 2 is a graph depicting the time-series plot of tumor size in the four treatment arms. The effects of treatments were assessed with a repeated measures ANOVA model. Treatment with cisplatin polymer and radiation resulted in significantly reduced tumor size over time (p=0.03 and 0.001, respectively).

FIG. 3, comprising FIG. 3A through FIG. 3C, is a set of graphs demonstrating that DC-CCL21 cultured in the polymer is capable to producing CCL21 in vitro. (FIG. 3A) DC-CCL21 were grown directly on plates or in polymer at different initial densities as indicated. Growth curve were made using cell numbers courted five days later (open bar). (FIG. 3B) Time dependent continuous release of CCL21 from DC-CCL21 in polymer. (FIG. 3C) Density dependent release of CCL21 from DC-CCL21 in polymer.

FIG. 4 is a graph demonstrating that polymer-based DC-CCL21 treatment inhibits tumor growth in a partially resected HNSCC model. Established SCCVII/SF tumors were partially resected and were treated with control, polymer, polymer+CCL21 injection, polymer+DC and polymer+DC-CCL21. Polymer+DC-CCL21 treatment exhibited a significant antitumor effect, starting from day 6 to day 12 (p<0.05). In contrast, polymer with CCL21 injection only demonstrated a weak and insignificant decrease in tumor growth, compared to control group or plain polymer group (p>0.05). Plain polymer or polymer+DC treatment showed no significant difference in tumor volume compared to the control group (p>0.05). Results are representative of three independent experiments; bar, ±SD.

FIG. 5 is a graph demonstrating that DC-CCL21 polymer recruits dendritic cells and T cells and inhibits regulatory T cells in vivo. Flow cytometry was performed to evaluate single cell suspensions of tumor nodules. Animals receiving DC-CCL21 polymer therapy exhibited a significant increase in the frequency of CD4+ T cell and CD11c+ dendritic cells, as well as a marked decrease in CD4+ CD25+ regulatory T cells infiltrating the tumor site.

FIG. 6 depicts the results of experiments demonstrating that DC-CCL21 treatment inhibits EMT in squamous cell tumors. Epithelial markers, including E-cadherin, beta-catenin and gamma-catenin, are increased, whereas the mesenchymal marker vimentin is decreased in the tumors from the DC-CCL21 polymer treatment group, as compared to tumors from the plain polymer control group.

FIG. 7, comprising FIG. 7A through FIG. 7C, is a set of graphs demonstrating that cisplatin secreting polymer enhances the efficacy of radiation therapy. (FIG. 7A) Time-course of tumor size of tumors treated with cisplatin polymer, control polymer, cisplatin polymer+radiation, and radiation alone. (FIG. 7B). Tumor size for tumors treated with cisplatin polymer or cisplatin polymer+radiation, using different radiation doses. (FIG. 7C) Time course of tumor size for tumors treated with cisplatin polymer or cisplatin polymer+radiation, using different radiation doses.

FIG. 8 is a graph depicting the CCL21 release kinetics from polymer.

FIG. 9 is a graph demonstrating that concomitant CCL21 and cisplatin secreting polymer further reduced tumor burden.

FIG. 10 is a schematic of an exemplary cisplatin and CCL21 releasing polymer of the present invention.

### DETAILED DESCRIPTION

The invention provides compositions and methods for delivering a therapeutic agent to a desired site for the treatment of cancer. In one embodiment, the invention provides a novel polymer platform to deliver a desired therapeutic agent to a site in need thereof for the treatment of cancer. In one embodiment, the polymer platform can be implanted at the tumor site. In another embodiment, the polymer platform can be implanted in proximity of the tumor. In one embodiment, the polymer platform provides a benefit to the subject by at least increasing the dosage of local therapy and by serving as a platform for immunomodulation/drug therapy.

In one embodiment, the novel polymer platform of the invention can be used to effectively grow cells. For example, dendritic cells can be grown in the polymer whereby the cells are able to secrete a therapeutic agent.

In one embodiment, the invention provides a drug delivery system comprising a therapeutic agent and a biodegradable polymer. In one embodiment, the drug delivery system of the present invention comprises a cell and a biodegradable polymer. In yet another embodiment, the invention provides a drug delivery system comprising a therapeutic agent, a cell and a biodegradable polymer.

In one embodiment, the invention provides a scaffold comprising one or more of a therapeutic agent and a cell. In one embodiment, the scaffold is an implantable biodegradable polymer. In one embodiment, the implantable biodegradable polymer is able to release one or more therapeutic agents into the implanted region. For example, in one embodiment, the implantable biodegradable polymer releases a therapeutic agent secreted by a cell within the polymer. In another embodiment, the implantable biodegradable polymer is able to release the one or more therapeutic agents and a cell into the implanted region. Therefore, in one embodiment, the invention provides a method of locally controlling the delivery of one or more therapeutic agents and a cell for an effective strategy for the treatment of cancer. In one embodiment, the method comprises profiling the tumor of the subject and designing a personalized polymer platform that is specifically tuned to treating the tumor the subject. For example, the polymer platform can be designed with specific types and amounts of therapeutic agents, release profiles, and polymer degradation characteristics.

In another embodiment, the compositions of the invention act to sensitize tumors to other forms of therapy, including but not limited to chemotherapy, radiation, hypothermia, and the like. In another embodiment, the polymer platform of the invention can direct the highest dose of radiation therapy to the tumor and spare surrounding normal tissues.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of for example ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“An antigen presenting cell” (APC) is a cell that is capable of activating T cells, and includes, but is not limited to, monocytes/macrophages, B cells and dendritic cells (DCs).

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

The term “biodegradable” includes polymers, compositions and formulations, such as those described herein, that are intended to degrade during use. Biodegradable polymers typically differ from non-biodegradable polymers in that the former may be degraded during use. In one embodiment, such use involves in vivo use, such as in vivo therapy. In another embodiment, such use involves in vitro use. In general, biodegradation involves the degradation of a biodegradable polymer into its component subunits, or digestion, e.g., by a biochemical process, of the polymer into smaller, non-polymeric subunits. Two types of biodegradation may generally be identified. For example, biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. Further, biodegradation may involve cleavage of a bond (whether covalent or otherwise) internal to side chain or that connects a side chain to the polymer backbone. For example, a therapeutic agent or other chemical moiety attached as a side chain to the polymer backbone may be released by biodegradation. In one embodiment, at least one type of biodegradation may occur during use of a polymer. As used herein, the term “biodegradation” encompasses all known types of biodegradation.

As used herein, the terms “biocompatible polymer” and “biocompatibility” when used in relation to polymers are recognized in the art. For example, biocompatible polymers include polymers that are generally neither toxic to the host, nor degrade (if the polymer degrades) at a rate that produces monomeric or oligomeric subunits or other byproducts at toxic concentrations in the host. In one embodiment, biodegradation generally involves degradation of the polymer in a host, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in one embodiment, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions be biocompatible as set forth above. Hence, a subject composition may comprise polymers comprising 99%, 98%, 97%, 96%, 95%, 90%, 85%, 80%, 75% or even less of biocompatible polymers, e.g., including polymers and other materials and excipients described herein, and still be biocompatible.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, head and neck cancers, lymphoma, leukemia, lung cancer and the like.

The term “dendritic cell” or “DC” refers to any member of a diverse population of morphologically similar cell types found in lymphoid or non-lymphoid tissues. These cells are characterized by their distinctive morphology, high levels of surface MHC-class II expression, and ability to regulate the immune response. DCs can be isolated from a number of tissue sources. DCs have a high capacity for sensitizing MHC-restricted T cells and are very effective at presenting antigens to T cells in situ. The antigens may be self-antigens that are expressed during T cell development and tolerance, and foreign antigens that are present during normal immune processes.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “gel” refers to a three-dimensional polymeric structure that itself is insoluble in a particular liquid but which is capable of absorbing and retaining large quantities of the liquid to form a stable, often soft and pliable, but always to one degree or another shape-retentive, structure. When the liquid is water, the gel is referred to as a hydrogel. Unless expressly stated otherwise, the term “gel” will be used throughout this application to refer both to polymeric structures that have absorbed a liquid other than water and to polymeric structures that have absorbed water, it being readily apparent to those skilled in the art from the context whether the polymeric structure is simply a “gel” or a “hydrogel.”

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of a composition includes, e.g., subcutaneous (s.c.), intravenous (i.v.), intramuscular (i.m.), or intrasternal injection, or infusion techniques. “Enteral” administration of a composition generally refers to delivery involving any part of gastrointestinal tract including oral delivery and rectal delivery. Parenteral and enteral administration have systemic effects.

As used herein, the term “polymer” refers to a molecule composed of repeating structural units typically connected by covalent chemical bonds. The term “polymer” is also meant to include the terms copolymer and oligomers.

As used herein, the term “polymerization” refers to at least one reaction that consumes at least one functional group in a monomeric molecule (or monomer), oligomeric molecule (or oligomer) or polymeric molecule (or polymer), to create at least one chemical linkage between at least two distinct molecules (e.g., intermolecular bond), at least one chemical linkage within the same molecule (e.g., intramolecular bond), or any combination thereof. A polymerization reaction may consume between about 0% and about 100% of the at least one functional group available in the system. In one embodiment, polymerization of at least one functional group results in about 100% consumption of the at least one functional group. In another embodiment, polymerization of at least one functional group results in less than about 100% consumption of the at least one functional group.

As used herein, the term “polymer segment” means and includes a grouping of multiple monomer units of a single type (i.e., a homopolymer segment) or multiple types (i.e., a copolymer segment) of constitutional units into a continuous region of a polymer block that are of a length that is insufficient for microphase separation to inherently occur with other segments in the same block type.

As used herein, the term “block copolymer” means and includes a polymer composed of chains where each chain contains two or more polymer blocks as defined above and at least two of the blocks are of sufficient segregation strength (e.g., χN>10) for those blocks to phase separate. A wide variety of block polymers are contemplated herein including diblock copolymers (i.e., polymers including two polymer blocks), triblock copolymers (i.e., polymers including three polymer blocks), multiblock copolymers (i.e., polymers including more than three polymer blocks), and combinations thereof.

As used herein, the term “patient,” “subject,” “individual,” and the like are used interchangeably herein, and refer to any animal, or cells thereof, whether in vitro or in situ, amenable to the methods described herein. In certain non-limiting embodiments, the patient, subject or individual is a mammal, non-limiting examples of which include a primate, dog, cat, goat, horse, pig, mouse, rat, rabbit, and the like, that is in need of bone formation. In some embodiments of the present invention, the subject is a human being. In such embodiments, the subject is often referred to as an “individual” or a “patient.” The terms “individual” and “patient” do not denote any particular age

As used herein, the phrase “a tumor site” refers to any site or region within a subject which a tumor has formed, may be expected to form, or was previously located. In certain embodiments, the tumor site is in need of anti-tumor activity.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention is based on the discovery that a novel polymer platform was successful in delivering one or more therapeutic agents (e.g., cisplatin and cytokines) to a partially resected squamous cell carcinoma (SCC) in an animal model. For example, a polymer secreting cisplatin was demonstrated to effectively reduce tumor size in the animal model. The invention is also based on the discovery that the novel polymer platform can be used to effectively grow dendritic cells whereby the cells actively secrete a therapeutic agent (e.g., CCL21). Accordingly, the invention provides a new therapeutic modality for treating cancer.

The present invention includes novel polymer platform that can be used as a drug delivery system comprising one or more therapeutic agents. In one embodiment, the invention includes a drug delivery system that facilitates a controlled release of one or more therapeutic agents to a local area. In another embodiment, the present invention includes a drug delivery system that facilitates sustained release of one or more therapeutic agents to a local area. In one embodiment, at least one of the one or more therapeutic agents released by the polymer platform is secreted by a cell residing within the polymer. The present invention further includes methods of making and using the polymer platform of the invention.

In one embodiment, the invention provides a method of treating a tumor in a subject, the method comprises inserting the polymer platform of the invention into a site in need thereof. In another embodiment, the polymer platform of the invention is inserted in proximity of the tumor site.

In one embodiment, the use of the polymer platform of the invention is biocompatible and degradable and therefore can serve as a platform to deliver therapeutic agents (e.g., immunomodulators and chemotherapeutic agents) and/or cells so as to most effectively kill tumor cells in the proximity of the polymer application. In one embodiment, the polymer platform is designed to be applied intraoperatively to the surgical bed after removing or debulking the tumor, thus allowing for enhanced post-operative treatment (e.g. radiation treatment), and also functioning as a platform for the delivery of therapeutic agents.

The invention provides compositions and methods for treating head and neck squamous cell carcinoma (HNSCC), or its associated premalignant lesions in a subject. Therapeutic compounds and/or cells are administered prophylactically or therapeutically in the context of the polymer platform to the invention to a subject suffering from at risk of (or susceptible to) developing HNSCC. Such subjects are identified using standard clinical methods.

In one embodiment, the polymer platform comprises a matrix, wherein the matrix comprises a blend of poly-ε-caprolactone (PCL) and poly(DL-lactide-co-ε-caprolactone) (PLCL). The ratio of PLCL:PCL may be optimized for desired mechanical, structural, or degradation properties of the matrix. In one embodiment, the ratio of PLCL:PCL is about 70:30. In one embodiment, the polymer matrix is constructed by dissolving PLCL and PCL in a suitable solvent (e.g., choloroform), and spreading the solution over a surface to form thin sheets.

### Polymer Platform

The polymer platform of the invention provides a safer and simpler alternative to current cytokine immunotherapies designed to deliver cytokines into the tumor microenvironment in a sustained manner. However, the invention should not be limited to delivery of cytokines. Rather, delivery of any therapeutic agent and/or cell is included in the invention. For example, the polymer platform of the invention can be used to deliver one or more therapeutic agents to a desired site in the mammal. In one embodiment, at least one of the one or more therapeutic agents released by the polymer platform is secreted by a cell residing within the polymer. In one embodiment, at least one of the one or more therapeutic agents released by the polymer platform is a cell.

In one embodiment, the polymer platform of the invention is a flexible sheet that is designed to be applied intraoperatively to the surgical bed after removing or debulking the tumor, and is engineered to adapt and adhere to the surgical resected tissue contours. The local delivery of a therapeutic agent using the polymer platform of the invention allows for maximizing therapeutic index, minimizing systemic side effects, and enhancing post-operative treatment (e.g. radiation treatment). In one embodiment, the polymer platform of the invention allows for a more durable sustained release of the therapeutic agent thereby increasing the interaction time with the target cells.

In one embodiment, the polymer platform of the invention provides a mechanical barrier to a tumor at the implant site. For example, the polymer platform prevents initial metastasis and angiogenesis of the tumor.

In one embodiment, the polymer platform of the invention provides a mechanical barrier to healthy tissues at the implant site. For example, in one embodiment, the polymer platform prevents or reduces the dose of anti-tumor agents (e.g., chemotherapeutic agents) that enter healthy tissue.

In one embodiment, the polymer platform serves as a radiosensitizer. For example, in certain embodiments, the polymer platform releases a therapeutic agent (e.g., cisplatin) to the tumor microenvironment which sensitizes the local tumor microenvironment to radiation. This allows for delivery of a lethal dose of radiation therapy while sparing nearby healthy tissues. For example, in one embodiment, the release of the radiosensitizer allows for a low dose of radiation therapy, that otherwise would not be able to effectively treat.

In one embodiment, the polymer platform comprises one or more protective agents, for example radioprotective agents, that can be delivered to healthy tissue to protect the healthy tissue during subsequent treatment of the tumor (e.g. radiation treatment).

In one embodiment, the polymer platform of the invention comprises defined regions which either comprise radiosensitizing agents to be delivered to tumor tissue or radioprotective agents to be delivered to healthy tissue. Thus, the platform may allow for specific delivery of sensitizing agents to tumor regions targeted by subsequent radiation therapy, while delivering protective agents to healthy regions to be spared damage during subsequent radiation therapy.

In one embodiment, the polymer platform of the invention is biocompatible and therefore is able to be used to culture cells. In one embodiment, the polymer platform of the invention comprises a desired cell and this cellular polymer platform can be used to treat cancer. In one embodiment, dendritic cells can be cultured with the polymer platform and the cellular polymer platform can be administered to the mammal to elicit an antitumor activity. For example, the dendritic cells present on the polymer platform secrete a therapeutic agent (e.g. CCL21) and the therapeutic agent can exhibit an antitumor activity.

In one embodiment, the polymer platform of the invention is a multi-layer polymer platform. For example, in certain embodiments, the polymer platform comprises different layers, each comprising a different therapeutic agent, combination of therapeutic agents, concentration of a therapeutic agent, degradation profile, porosity, or the like.

In one embodiment, the platform comprises a chemotherapeutic layer and an immunomodulator layer.

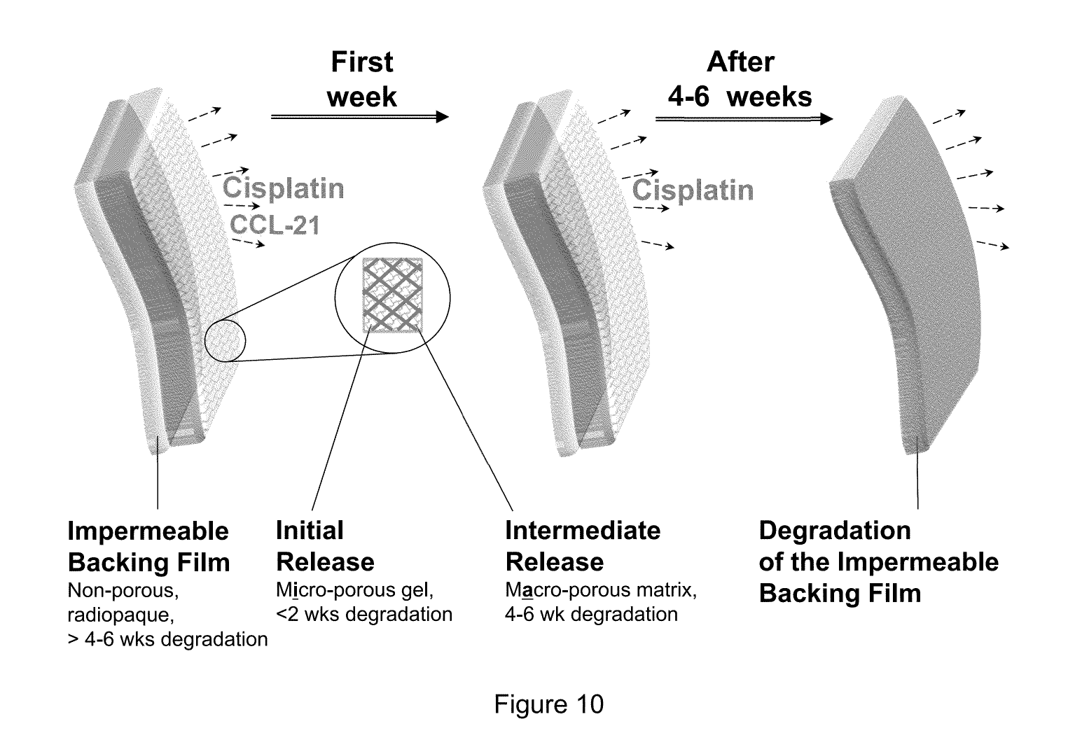

For example, in one embodiment, the platform comprises a hydrogel layer and a polymer matrix layer (FIG. 10). In one embodiment, the hydrogel layer comprises an immumomodulator. For example, in one embodiment, the hydrogel layer comprises a cell modified to secrete an immunomodulator. In one embodiment, the hydrogel layer comprises a dendritic cell modified to secrete CCL21. In one embodiment, the hydrogel layer comprises an immunomodulator and a chemotherapeutic agent. In one embodiment, the polymer matrix layer comprises a chemotherapeutic agent. The degradation properties of the different layers of the platform dictate the release profile of the one or more therapeutic agents. For example, in one embodiment, the hydrogel layer releases the one or more therapeutic agents contained therein within about 2 weeks post implantation. In one embodiment, the polymer matrix layer releases the one or more therapeutic agents contained therein within 4-6 weeks post implantation.

In one embodiment, the platform comprises an impermeable backing layer or film, which prevents release of the one or more therapeutic agents through the backing layer. For example, in one embodiment, the backing layer prevents the delivery of potentially harmful agents to healthy tissue, thereby sparing healthy tissue while the polymer platform delivers the agents to the tumor site. In certain embodiments, the backing layer comprises one or more tissue protective agents that protect the healthy tissue, including for example radioprotective agents. In certain embodiments, the impermeable backing layer aids in the local delivery of the one or more therapeutic agents to the tumor site. In one embodiment, the backing layer is degradable, degrading after about 4-6 weeks post implantation.

In one embodiment, one or more layers of the polymer platform of the invention is radiopaque, allowing for the detection of the polymer platform when implanted. In certain embodiments, the radiopaque polymer platform allows for the observation of tumor size, polymer, degradation, and the like. For example, in certain embodiments, one or more layers of the platform are radiopaque. The polymer platform of the invention may be made radiopaque via the inclusion of radiopaque agents into the platform. Exemplary radiopaque agents include, but are not limited to barium sulfate, tantalum, and the like.

In one embodiment, the polymer platform of the invention is modular, which allows for the tailoring or customizing the platform for treatment of individual patents or individual tumors. For example, the type of therapeutic agents, concentration of the therapeutic agents, degradation properties of the platform, presence and location of protective agents, and the like may be specifically customized to the needs of the patient. In certain embodiments, the platform is customized based upon the size of the tumor, geometry of the tumor, type of tumor, location of tumor, age of the subject, a biomarker profile, drug sensitivity of the tumor or the like. In certain embodiments, the polymer platform is formulated according to a series of common sizes and geometries. In one embodiment, the polymer platform is custom fabricated based on the patient's individual 3-D medical image data. The modular nature of the polymer platform allows for altering the properties of one layer of the platform, without altering any other layer. Thus, unique multi-layer platforms may be designed and customized to best treat the specific tumor.

In one embodiment, the invention comprises a second polymer platform which provides a mechanical barrier to healthy tissues at the implant site. For example, the polymer platform prevents or reduces the dose of anti-tumor agents that enter healthy tissues. This polymer may be applied manually in situ, or pre-fabricated into a common size or custom size. The polymer may also contain fillers in strategic locations that attenuate ionizing radiation to minimize damage to healthy tissues.

### Polymer Matrix

In one embodiment, the present invention provides a drug delivery system comprising a polymer matrix comprising one or more therapeutic agents. In one embodiment, the present invention provides a polymer platform comprising a polymer matrix layer, which comprises at least one or more releasable therapeutic agents.

The one or more therapeutic agents of the invention may be used in amounts that are therapeutically effective. The amount of the one or more therapeutic agents incorporated into the polymer matrix also depends upon the desired release profile, the concentration of the one or more therapeutic agents required for a biological effect, and the length of time that the one or more therapeutic agents has to be released for treatment. In one embodiment, the one or more therapeutic agents may be blended with a polymer matrix at different loading levels, in one embodiment at room temperature and without the need for an organic solvent.

There is no critical upper limit on the amount of the one or more therapeutic agents incorporated except for that of an acceptable solution or dispersion viscosity to maintain the physical characteristics desired for the composition. The lower limit of the one or more therapeutic agents incorporated into the polymer system is dependent upon the activity of the one or more therapeutic agents and the length of time needed for treatment. Thus, the amount of the one or more therapeutic agents should not be so small that it fails to produce the desired physiological effect, nor so large that the agent is released in an uncontrollable manner. Typically, within these limits, amounts of the one or more therapeutic agents from about 1% up to about 60% may be incorporated into the present delivery systems. However, lesser amounts may be used to achieve efficacious levels of treatment for one or more therapeutic agents that is particularly potent.

In one embodiment, the polymer matrix of the platform can be considered a type of scaffold or polymeric implant. That is, the polymer matrix may be used as the drug depot. In some instances, the drug is a chemotherapeutic agent. In one embodiment, precursor is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the encapsulated drug and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the one or more therapeutic agents can be incorporated into a polymer matrix and molded or compressed into a device that is a solid at room temperature. For example, the one or more therapeutic agents can be incorporated into a biodegradable polymer, such as polyanhydrides and copolymers thereof, polyhydroalkanoic acids and copolymers thereof, PLA, PGA, and PLGA, and compressed into solid device, such as sheets, disks, or extruded into a device, such as rods.

In one embodiment, the polymer matrix is sufficiently hydrophobic so that it retains its integrity for a suitable period of time when placed in an aqueous environment, such as the body, and stable enough to be stored for an extended period before use. The polymer matrix should provide a suitable degradation profile, so that it remains in the patient's body for a suitable period of time to release the one or more therapeutic agents contained therein, while degrading into biocompatible degradation products. The polymer matrix should be sufficiently strong and flexible so that it does not crumble or fragment during use.

Biocompatible polymers can be categorized as biodegradable and non-biodegradable. Biodegradable polymers degrade in vivo as a function of chemical composition, method of manufacture, and implant structure. Synthetic and natural polymers can be used although synthetic polymers are preferred due to more uniform and reproducible degradation and other physical properties. Examples of synthetic polymers include polyanhydrides, polyhydroxyacids such as polylactic acid, polyglycolic acids and copolymers thereof, polyesters, polyamides, polyorthoesters, and some polyphosphazenes. Examples of naturally occurring polymers include proteins and polysaccharides such as collagen, hyaluronic acid, albumin and gelatin. The one or more therapeutic agents can be encapsulated within, throughout, and/or on the surface of the matrix.

There are two general classes of biodegradable polymers: those degrading by bulk erosion and those degrading by surface erosion. As a non-limiting example, an aromatic monomer such as p-carboxyphenoxy propane (CPP) may be copolymerized with a monomer such as sebacic acid (SA) to form a copolymer, such as CPP-SA (20:80).

Use of polyanhydrides in controlled delivery devices has been reported by Leong, et al., J. Med. Biomed. Mater. Res. 19, 941 (1985); J. Med. Biomed. Mater. Res. 20, 51 (1986); and Rosen, et al., Biomaterials 4, 131 (1983). The release and physical properties required for processing into implants are largely determined by the hydrophobicity and molecular weight, with higher molecular weight polymers having more desirable physical properties. Aromatic polyanhydrides exhibit near zero order (linear) erosion and release kinetics, but have very slow degradation rates. For example, it was estimated that it would take a delivery device prepared from p-CPP more than three years to completely degrade in vivo. Polymers prepared from linear aliphatic diacids are hydrophilic solids that degrade by bulk erosion, resulting in a rapid release of the drug from the polymeric matrix. Further, anhydride homopolymers based on aromatic or linear aliphatic dicarboxylic acids are highly crystalline and have poor film forming properties. Aromatic polyanhydrides also have high melting points and low solubility in organic solvents. Copolymerizing the linear aliphatic diacids with aromatic diacids, to form, for example, the copolymer of poly 1,3-(bis(p-carbophenoxy)propane anhydride (p-CPP) (an aromatic polyanhydride) with sebacic acid (a copolymer of an aromatic diacid and an aliphatic diacid), can be used to obtain polymers having appropriate degradation times. As described in U.S. Pat. No. 4,757,128 to Domb and Langer, high molecular weight copolymers of aliphatic dicarboxylic acids with aromatic diacids are less crystalline than aromatic or linear aliphatic polyanhydrides, and they form flexible films. U.S. patents that describe the use of polyanhydrides for controlled delivery of substances include U.S. Pat. No. 4,857,311 to Domb and Langer, U.S. Pat. No. 4,888,176 to Langer, et al., and U.S. Pat. No. 4,789,724 to Domb and Langer.

Other polymers such as polylactic acid, polyglycolic acid, and copolymers thereof have been commercially available as suture materials for a number of years and can be readily formed into devices for drug delivery.

In one embodiment, the substrate is a polymer comprising a synthetic polymer or copolymer prepared from at least one of the group of monomers consisting of acrylic acid, methacrylic acid, ethyleneimine, crotonic acid, acrylamide, ethyl acrylate, methyl methacrylate, 2-hydroxyethyl methacrylate, lactic acid, glycolic acid, .ε-caprolactone, acrolein, cyanoacrylate, bisphenol A, epichlorhydrin, hydroxyalkylacrylates, siloxane, dimethylsiloxane, ethylene oxide, ethylene glycol, hydroxyalkyl-methacrylates, N-substituted acrylamides, N-substituted methacrylamides, N-vinyl-2-pyrrolidone, 2,4-pentadiene-1-ol, vinyl acetate, acrylonitrile, styrene, p-amino-styrene, p-amino-benzyl-styrene, sodium styrene sulfonate, sodium 2-sulfoxyethyl methacrylate, vinyl pyridine, aminoethyl methacrylates, 2-methacryloyloxy-trimethylammonium chloride, N,N′-methylenebisacrylamide-, ethylene glycol dimethacrylates, 2,2′-(p-phenylenedioxy)-diethyl dimethacrylate, divinylbenzene, and triallylamine, methylenebis-(4-phenyl-isocyanate).

A variety of polymers from synthetic and/or natural sources can be used to produce the polymer matrix of the polymer platform of the invention. For example, lactic or polylactic acid or glycolic or polyglycolic acid can be utilized to form poly(lactide) (PLA) or poly(L-lactide) (PLLA) nanofibers or poly(glycolide) (PGA) nanofibers. The polymer matrix can also be made from more than one monomer or subunit thus forming a co-polymer, terpolymer, etc. For example, lactic or polylactic acid and be combined with glycolic acid or polyglycolic acid to form the copolymer poly(lactide-co-glycolide) (PLGA). Other copolymers of use in the invention include poly(ethylene-co-vinyl) alcohol). In an exemplary embodiment, the polymer matrix can comprise a polymer or subunit which is a member selected from an aliphatic polyester, a polyalkylene oxide, polydimethylsiloxane, polyvinylalcohol, polylysine, and combinations thereof. In another exemplary embodiment, the polymer matrix can comprises two different polymers or subunits which are members selected from an aliphatic polyester, a polyalkylene oxide, polydimethylsiloxane, polyvinylalcohol, polylysine, and combinations thereof. In another exemplary embodiment, the polymer matrix comprises three different polymers or subunits which are members selected from an aliphatic polyester, a polyalkylene oxide, polydimethylsiloxane, polyvinylalcohol, polylysine, and combinations thereof. In an exemplary embodiment, the aliphatic polyester is linear or branched. In another exemplary embodiment, the linear aliphatic polyester is a member selected from lactic acid (D- or L-), lactide, poly(lactic acid), poly(lactide) glycolic acid, poly(glycolic acid), poly(glycolide), glycolide, poly(lactide-co-glycolide), poly(lactic acid-co-glycolic acid), polycaprolactone and combinations thereof. In another exemplary embodiment, the aliphatic polyester is branched and comprises at least one member selected from lactic acid (D- or L-), lactide, poly(lactic acid), poly(lactide) glycolic acid, poly(glycolic acid), poly(glycolide), glycolide, poly(lactide-co-glycolide), poly(lactic acid-co-glycolic acid), polycaprolactone and combinations thereof which is conjugated to a linker or a biomolecule. In an exemplary embodiment, wherein said polyalkylene oxide is a member selected from polyethylene oxide, polyethylene glycol, polypropylene oxide, polypropylene glycol and combinations thereof.

As another example, polymer matrix may be formed from functionalized polyester graft copolymers. The functionalized graft copolymers are copolymers of polyesters, such as poly(glycolic acid) or poly(lactic acid), and another polymer including functionalizable or ionizable groups, such as a poly(amino acid). In another embodiment, polyesters may be polymers of α-hydroxy acids such as lactic acid, glycolic acid, hydroxybutyric acid and valeric acid, or derivatives or combinations thereof. The inclusion of ionizable side chains, such as polylysine, in the polymer has been found to enable the formation of more highly porous particles, using techniques for making microparticles known in the art, such as solvent evaporation. Other ionizable groups, such as amino or carboxyl groups, may be incorporated, covalently or noncovalently, into the polymer to enhance porosity. For example, polyaniline could be incorporated into the polymer. These groups can be modified further to contain hydrophobic groups capable of binding load molecules.

In an exemplary embodiment, the polymer matrix can include one or more of the following: polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly-ε-caprolactone, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), poly(vinyl acetate), polyvinyl chloride, polystyrene, polyvinyl pyrrolidone, pluronics, polyvinylphenol, saccharides (e.g., dextran, amylose, hyalouronic acid, poly(sialic acid), heparans, heparins, etc.); poly (amino acids), e.g., poly(aspartic acid) and poly(glutamic acid); nucleic acids and copolymers thereof.

In an exemplary embodiment, the polymer matrix can include one or more of the following: peptide, saccharide, poly(ether), poly(amine), poly(carboxylic acid), poly(alkylene glycol), such as poly(ethylene glycol) (“PEG”), poly(propylene glycol) (“PPG”), copolymers of ethylene glycol and propylene glycol and the like, poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, poly(N-acryloylmorpholine), polysialic acid, polyglutamate, polyaspartate, polylysine, polyethyeleneimine, biodegradable polymers (e.g., polylactide, polyglyceride and copolymers thereof), polyacrylic acid.

In one embodiment, the polymer matrix comprises a blend of poly-ε-caprolactone (PCL) and poly(DL-lactide-co-ε-caprolactone) (PLCL). The ratio of PLCL:PCL may be optimized for desired mechanical, structural, or degradation properties of the matrix. In one embodiment, the ratio of PLCL:PCL is about 70:30. In one embodiment, the polymer matrix is constructed by dissolving PLCL and PCL in a suitable solvent (e.g., choloroform), and spreading the solution over a surface to form thin sheets.

### Hydrogels

In one embodiment, the polymer platform of the invention comprises a hydrogel. For example, in certain embodiments, the platform comprises a hydrogel comprising one or more therapeutic agents.

Hydrogels can generally absorb much fluid and, at equilibrium, typically are composed of 60-90% fluid and only 10-30% polymer. In one embodiment, the water content of hydrogel is about 70-80%. Hydrogels are particularly useful due to the inherent biocompatibility of the polymeric network (Hill-West, et al., 1994, Proc. Natl. Acad. Sci. USA 91:5967-5971). Hydrogel biocompatibility can be attributed to hydrophilicity and ability to imbibe large amounts of biological fluids (Brannon-Peppas. Preparation and Characterization of Cross-linked Hydrophilic Networks in Absorbent Polymer Technology, Brannon-Peppas and Harland, Eds. 1990, Elsevier: Amsterdam, pp 45-66; Peppas and Mikos. Preparation Methods and Structure of Hydrogels in Hydrogels in Medicine and Pharmacy, Peppas, Ed. 1986, CRC Press: Boca Raton, Fla., pp 1-27). In certain embodiments, the hydrogels can be prepared by crosslinking hydrophilic biopolymers or synthetic polymers. In certain embodiments, construction of hydrogels comprises the polymerization and/or copolymerization of monomers, macromers, polymers and the like. For example, in one embodiment hydrogel formation comprises copolymerization of two or more types of biopolymers and/or synthetic polymers.

Hydrogels may be prepared by crosslinking hydrophilic biopolymers or synthetic polymers. Examples of the hydrogels formed from physical or chemical crosslinking of hydrophilic biopolymers include but are not limited to, hyaluronans, chitosans, alginates, collagen, dextran, pectin, carrageenan, polylysine, gelatins, fibrin, or agarose. Examples of hydrogels based on chemical or physical crosslinking synthetic polymers include but are not limited to (meth)acrylate-oligolactide-PEO-oligolactide-(meth)acrylate, poly(ethylene glycol) (PEO), polypropylene glycol) (PPO), PEO-PPO-PEO copolymers (Pluronics), poly(phosphazene), poly(methacrylates), poly(N-vinylpyrrolidone), PL(G)A-PEO-PL(G)A copolymers, or poly(ethylene imine).

In one embodiment, the hydrogel comprises at least one biopolymer. In other embodiments, the hydrogel comprises at least two biopolymers. In yet other embodiments, the hydrogel comprises at least one biopolymer and at least one synthetic polymer.

Hydrogels closely resemble the natural living extracellular matrix (Ratner and Hoffman. Synthetic Hydrogels for Biomedical Applications in Hydrogels for Medical and Related Applications, Andrade, Ed. 1976, American Chemical Society: Washington, D.C., pp 1-36). Hydrogels can also be made degradable in vivo by incorporating PLA, PLGA or PGA polymers. Moreover, hydrogels can be modified with fibronectin, laminin, vitronectin, or, for example, RGD for surface modification, which can promote cell adhesion and proliferation (Heungsoo Shin, 2003, Biomaterials 24:4353-4364; Hwang et al., 2006 Tissue Eng. 12:2695-706). Indeed, altering molecular weights, block structures, degradable linkages, and cross-linking modes can influence strength, elasticity, and degradation properties of the instant hydrogels (Nguyen and West, 2002, Biomaterials 23(22):4307-14; Ifkovits and Burkick, 2007, Tissue Eng. 13(10):2369-85).

Molecules which can be incorporated into the hydrogel, for example via covalent linkage, encapsulation, or the like, include, but are not limited to, vitamins and other nutritional supplements; glycoproteins (e.g., collagen); fibronectin; peptides and proteins; carbohydrates (both simple and/or complex); proteoglycans; antigens; oligonucleotides (sense and/or antisense DNA and/or RNA); antibodies (for example, to infectious agents, tumors, drugs or hormones); contrast agents; radiopaque agents; and gene therapy reagents. Hydrogels may be modified with functional groups for covalently attaching a variety of proteins (e.g., collagen) or compounds such as therapeutic agents. Therapeutic agents which can be incorporated to the matrix include, but are not limited to, analgesics, anesthetics, antifungals, antibiotics, anti-inflammatories, anthelmintics, antidotes, antiemetics, antihypertensives, antimalarials, antimicrobials, antipsychotics, antipyretics, antiseptics, antituberculotics, antivirals, cardioactive drugs, chemotherapeutic agents, a colored or fluorescent imaging agent, corticoids (such as steroids), antidepressants, depressants, diagnostic aids, enzymes, hormones, hypnotics, minerals, nutritional supplements, parasympathomimetics, potassium supplements, radiation sensitizers, a radioisotope, sedatives, sulfonamides, stimulants, sympathomimetics, tranquilizers, vasoconstrictors, vasodilators, vitamins, xanthine derivatives, and the like. The therapeutic agent can also be other small organic molecules, naturally isolated entities or their analogs, organometallic agents, chelated metals or metal salts, peptide-based drugs, or peptidic or non-peptidic receptor targeting or binding agents. It is contemplated that linkage of the therapeutic agent to the hydrogel can be via a protease sensitive linker or other biodegradable linkage.

In certain embodiments, one or more multifunctional cross-linking agents may be utilized as reactive moieties that covalently link biopolymers or synthetic polymers. Such bifunctional cross-linking agents may include glutaraldehyde, epoxides (e.g., bis-oxiranes), oxidized dextran, p-azidobenzoyl hydrazide, N-[α.-maleimidoacetoxy]succinimide ester, p-azidophenyl glyoxal monohydrate, bis-[β-(4-azidosalicylamido)ethyl]disulfide, bis[sulfosuccinimidyl]suberate, dithiobis[succinimidyl proprionate, disuccinimidyl suberate, 1-ethyl-3-[3-dimethylaminopropyl]carbodiimide hydrochloride (EDC), N-hydroxysuccinimide (NHS) and other bifunctional cross-linking reagents known to those skilled in the art. It should be appreciated by those in skilled in the art that the mechanical properties of the hydrogel are greatly influenced by the cross-linking time and the amount of cross-linking agents.

In another embodiment utilizing a cross-linking agent, polyacrylated materials, such as ethoxylated (20) trimethylpropane triacrylate, may be used as a non-specific photo-activated cross-linking agent. Components of an exemplary reaction mixture would include a thermoreversible hydrogel held at 39° C., polyacrylate monomers, such as ethoxylated (20) trimethylpropane triacrylate, a photo-initiator, such as eosin Y, catalytic agents, such as 1-vinyl-2-pyrrolidinone, and triethanolamine. Continuous exposure of this reactive mixture to long-wavelength light (>498 nm) would produce a cross-linked hydrogel network.

In one embodiment, the hydrogel comprises a UV sensitive curing agent which initiates hydrogel polymerization. For example, in one embodiment, a hydrogel comprises the photoinitiator 4-(2-hydroxyethoxy)phenyl-(2-hydroxy-2-propyl)ketone. In one embodiment, polymerization is induced by 4-(2-hydroxyethoxy)phenyl-(2-hydroxy-2-propyl)ketone upon application of UV light. Other examples of UV sensitive curing agents include 2-hydroxy-2-methyl-1-phenylpropan-2-one, 4-(2-hydroxyethoxy)phenyl (2-hydroxy-2-phenyl-2-hydroxy-2-propyl)ketone, 2,2-dimethoxy-2-phenyl-acetophenone 1-[4-(2-Hydroxyethoxy)-phenyl]-2-hydroxy-2-methyl-1-propane-1-one, 1-hydroxycyclohexylphenyl ketone, trimethyl benzoyl diphenyl phosphine oxide and mixtures thereof.

In one embodiment, the hydrogel comprises fibrin. In certain embodiments, fibrin hydrogels are formed by a modified polycondensation reaction from fibrinogen, as mediated by the cleavage of fibrinogen by the protease thrombin (Jamney et al., 2009, J. R. Soc. Interface, 6(30): 1-10). For example, thrombin may be added to a solution comprising fibrinogen in order to induce fibrin polymerization and thereby form a fibrin hydrogel.

In one embodiment, the hydrogel may be further stabilized and enhanced through the addition of one or more enhancing agents. The term “enhancing agent” or “stabilizing agent” refers to any compound added to the hydrogel scaffold, in addition to the high molecular weight components, that enhances the hydrogel scaffold by providing further stability or functional advantages. The enhancing agent may include any compound, such as polar compounds, that enhance the hydrogel by providing further stability or functional advantages when incorporated in the cross-linked hydrogel.

Preferred enhancing agents for use with hydrogel include polar amino acids, amino acid analogues, amino acid derivatives, intact collagen, and divalent cation chelators, such as ethylenediaminetetraacetic acid (EDTA) or salts thereof. Polar amino acids include tyrosine, cysteine, serine, threonine, asparagine, glutamine, aspartic acid, glutamic acid, arginine, lysine, or histidine. In one embodiment, the contemplated polar amino acids are L-cysteine, L-glutamic acid, L-lysine, and L-arginine. Polar amino acids, EDTA, and mixtures thereof, are also contemplated enhancing agents. The enhancing agents may be added to the hydrogel before or during the crosslinking of the high molecular weight components. The hydrogel may exhibit an intrinsic bioactivity, which may be a function of the unique stereochemistry of the cross-linked macromolecules in the presence of the enhancing and strengthening polar amino acids, as well as other enhancing agents.

In certain embodiments, the hydrogel is modified to improve its functionality. For example, the hydrogel may be coated with any number of compounds in order enhance its biocompatibility, reduce its immunogenicity, enhance stability, enhance degradation, and/or enhance drug delivery.

### Therapeutic Agent

The novel polymer platform of the invention may be used in combination with or include one or more therapeutic agents and may be administered by any convenient route. The novel polymer platform comprising a therapeutic agent is useful for preventing and/or treating cancer. Any therapeutic agent can be used in the context of the novel polymer platform of the invention. For example, in certain embodiments, the one or more therapeutic agents delivered by the polymer platform of the invention include, but is not limited to, proteins, peptides, hormones, vitamins, nutritional supplements, antigens, oligonucleotides (sense and/or antisense DNA and/or RNA), antibodies (for example, to infectious agents, tumors, drugs or hormones), cells, chemotherapeutic agents, radiosenstizers, and gene therapy reagents. Alternatives to monoclonal antibodies include single-chain variable fragments, minibodies, tetrabodies, tribodies, diabodies, and in vitro selected antibodies.

In one embodiment, the one or more therapeutic agents of the platform include any anti-tumor agent, including but not limited to a chemotherapeutic agent, an anti-cell proliferation agent, radiosensitizing agent, or any combination thereof. For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents.

Alkylating agents are so named because of their ability to add alkyl groups to many electronegative groups under conditions present in cells, thereby interfering with DNA replication to prevent cancer cells from reproducing. Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Anti-metabolites prevent incorporation of bases into DNA during the synthesis (S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

Antitumor antibiotics generally prevent cell division by interfering with enzymes needed for cell division or by altering the membranes that surround cells.

Included in this class are the anthracyclines, such as doxorubicin, which act to prevent cell division by disrupting the structure of the DNA and terminate its function. These agents are cell cycle non-specific. Non-limiting examples of antitumor antibiotics include dactinomycin, daunorubicin, doxorubicin, idarubicin, mitomycin-C, and mitoxantrone.

Plant alkaloids inhibit or stop mitosis or inhibit enzymes that prevent cells from making proteins needed for cell growth. Frequently used plant alkaloids include vinblastine, vincristine, vindesine, and vinorelbine. However, the invention should not be construed as being limited solely to these plant alkaloids.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to therapy. Some other tumors are also hormone dependent.

Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine that are also useful in the invention. An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In one embodiment, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In another embodiment, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, saporin 6, or a combination thereof.

In one embodiment, the novel polymer platform of the invention can be used to deliver a therapeutic agent such as an anticancer agent. An anticancer agent includes but is not limited to everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a PD-1 inhibitor, a Cdk inhibitor, an EGFR TK inhibitor, an IGFR-TK inhibitor, an anti-HGF antibody, an anti-CD47 antibody, an anti-GD2 antibody, an anti-EGF receptor antibody, a PI3 kinase inhibitors, an AKT inhibitor, a JAK/STAT inhibitor, immune checkpoint blockades, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR1 KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, pyro-Glu-His-Trp-Ser-Tyr-D-Ser(But)-Leu-Arg-Pro-Azgly-NH₂ x(acetate) wherein x=1 to 2.4, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

In one embodiment, at least one of the one or more therapeutic agents delivered by the polymer platform of the invention comprises an immunomodulator. The immunomodulator may be any peptide, protein, oligonucleotide, antibody, small molecule, polysaccharide, or the like, which modulates the immune system of the subject to be treated. For example, in certain embodiments, the immunomodulator boosts the immune system of the subject to increase immune cell recruitment to the tumor site, tumor cell killing, or the like. Exemplary immunomodulators include, but are not limited to, CCL21, IL-2, IL-6, IL-8, IL-7, IL-10, IL-12, interferons, G-CSF, imiquimod, CCL3, CCL26, CXCL7, oligodeoxynucleotides, glucan. For example, it is demonstrated herein that delivery of CCL21 (secondary lymphoid chemokine, SLC) by the polymer platform of the invention reduces tumor burden.

In one embodiment, the one or more therapeutic agents being released, along with the polymer barriers, produce a three dimensional tumor-suppressive microenvironment that activate local immune cells to perform their immunoediting duties, and/or inhibit the soluble factors (e.g. NKG2D) that are actively secreted by tumor cells to inhibit immune cells (See, for example, Vesely et al., 2011, Annual Review of Immunology, 29: 235-271).

For example, in one embodiment, the one or more agents released by the polymer platform are inhibitors of the mechanisms by which cancer evade host response. For example, almost all cancers display CD47 on their surface to instruct the host immune cells, such as macrophages, to treat the cancer cells as normal cells, thereby evading direct innate immune response and the subsequent adaptive immune response. Thus, by using antibodies that inhibit CD47, it has been shown that both innate and adaptive immune systems can be activated to destroy tumors (Edris et al., 2012, Proc Natl Acad Sci USA, 109(17): 6656-6661)

In one embodiment, the polymer platform comprises microparticles or nanoparticles which may be released from the polymer. For example, in one embodiment, the particles may comprise a targeting moiety that allow for its homing specifically to tumor cells or tissue. Some particles may also be photothermally active, that is, in response to near infrared irradiation, they can generate heat that increases the permeability of the cancer cell barrier, and also accelerate the molecular diffusion of the therapeutic agents. Some particles may respond to focused ultrasound energy to promote similar activities. In one embodiment, the particles comprise one or more therapeutic agents which are then delivered to the tumor cells.

In one embodiment, at least one of the one or more therapeutic agents is an isolated protein or polypeptide. In certain embodiments, the isolated polypeptide has an anti-tumor effect. In one embodiment, the isolated polypeptide is an immunomodulator. For example, in one embodiment, the isolated polypeptide is CCL21.

Variants of suitable polypeptides of the invention can also be expressed. Variants may be made by, for example, the deletion, addition, or alteration of amino acids that have either (i) minimal influence on certain properties, secondary structure, and hydropathic nature of the polypeptide or (ii) substantial effect on one or more properties of the peptide mimetics of the invention.

Variants may also include, for example, a polypeptide conjugated to a linker or other sequence for ease of synthesis, purification, identification, or therapeutic use (i.e., delivery) of the polypeptide.

The variants of the polypeptide according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polyeptide is an alternative splice variant of the polypeptide of the present invention, (iv) fragments of the polypeptides and/or (v) one in which the polypeptide is fused with another peptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

In certain embodiments, the polypeptide of the platform may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

A polypeptide of the polymer platform may be synthesized by conventional techniques. For example, the polypeptide may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.)

The polypeptide may be chemically synthesized by Merrifield-type solid phase peptide synthesis. This method may be routinely performed to yield polypeptides up to about 60-70 residues in length, and may, in some cases, be utilized to make polypeptides up to about 100 amino acids long. Larger polypeptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

In one embodiment, at least one of the one or more therapeutic agents is an isolated nucleic acid encoding an isolated peptide. For example, in one embodiment, at least one therapeutic agent comprises an expression vector which may be released at the treatment site to genetically modify a cell and encode a therapeutic polypeptide.

In one embodiment, at least one of the one or more therapeutic agents is a cell. For example, in one embodiment, the cell is released by the polymer platform at the treatment site. In one embodiment, the cell secretes a therapeutic agent (e.g., an immunomodulator). For example, in one embodiment, the cell secretes CCL21. In certain embodiments, the cell is genetically modified, as described elsewhere herein.

### Cellular Therapy

The present invention relates to the discovery that the novel polymer platform can be used to culture cells (e.g., dendritic cells). The cells cultured on or in the polymer platform can secrete therapeutic agents to reduce growth of the tumor. The cells can be genetically modified to secrete the desired therapeutic agent. In this context, cells can be cultured in the polymer platform and be used as a form of protein therapy.

Accordingly, the present invention encompasses methods and compositions for culturing and genetically modifying cells. The cells of the invention can be generated by transducing the cells with a vector that results in increased expression of a desired molecule. Any of a variety of methods well known to one of skill in the art can be used to transduce the cells.

Various types of cells may be used in the present invention, including but not limited to dendritic cells, T-cells, stem cells, adipose stem cells, induced pluripotent stem cells, embryonic stem cells, adult stem cells, cord blood derived stem cells, and the like.

The invention includes a vector comprising an isolated nucleic acid encoding a desired molecule. The nucleic acid encoding the desired molecule is operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York).

The nucleic acid encoding the desired molecule can be cloned into a number of types of vectors. However, the present invention should not be construed to be limited to any particular vector. Instead, the present invention should be construed to encompass a wide plethora of vectors which are readily available and well-known in the art. For example, an isolated nucleic acid encoding an immunosuppressive molecule of the invention can be cloned into a vector including, but not limited to a plasmid, a phagemid, a phage derivative, an animal virus, and a cosmid. Vectors of particular interest include expression vectors, replication vectors, probe generation vectors, and sequencing vectors.

The expression vector may be provided to a cell in the form of a viral vector. Viral vector technology is well known in the art and is described, for example, in Sambrook et al. (2001) and in other virology and molecular biology manuals. Viruses, which are useful as vectors include, but are not limited to, retroviruses, adenoviruses, adeno-associated viruses, herpes viruses, and lentiviruses.

In order to assess the expression of the desired molecule, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction, sites. Constructs may then be transfected into cells that display high levels of the desired polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription. Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

The cells can be obtained from any source, for example, from the patient or an otherwise unrelated source (a different individual or species altogether). The cells may be autologous with respect to the recipient or allogeneic with respect to the recipient.

Cells can be suspended in an appropriate diluent, at a concentration of from about 0.01 to about 5×10⁶ cells/ml. Suitable excipients are those that are biologically and physiologically compatible with the cells and with the recipient, such as buffered saline solution or other suitable excipients. The composition for administration can be formulated, produced and stored according to standard methods complying with proper sterility and stability.

In certain embodiments, the cells are administered to a polymer precursor solution prior to the formation of the polymer platform of the invention. For example, in one embodiment, the cells are administered to a hydrogel precursor solution, prior to polymerization, solidification, or crosslinking, of the hydrogel.

The dosage of the cells varies within wide limits and may be adjusted to the mammal requirements in each particular case. The number of cells used depends on the weight and condition of the recipient, the number and/or frequency of administrations, and other variables known to those of skill in the art.

Between about 10⁵ and about 10¹³ cells per 100 kg body weight can be administered to the mammal. In some embodiments, between about 1.5×10⁶ and about 1.5×10¹² cells are administered per 100 kg body weight. In some embodiments, between about 1×10⁹ and about 5×10″ cells are administered per 100 kg body weight. In some embodiments, between about 4×10⁹ and about 2×10¹¹ cells are administered per 100 kg body weight. In some embodiments, between about 5×10⁸ cells and about 1×10¹⁰ cells are administered per 100 kg body weight.

It is contemplated that the cells in the context of the polymer platform of the present invention may be administered into a recipient as a “one-time” therapy for the treatment of cancer. A one-time administration of cells into the recipient eliminates the need for repeated administrations. However, if desired, multiple administrations of cells may also be employed.

Gene therapy can be used to replace genes that are defective in a mammal. The invention may also be used to express a desired protein in a mammal. A cell can be introduced with a gene for a desired protein and introduced into a mammal within whom the desired protein would be produced and exert or otherwise yield a therapeutic effect. This aspect of the invention relates to gene therapy in which therapeutic proteins are administered to a mammal by way of introducing a genetically modified cell into a mammal. The genetically modified cells in the context of the polymer platform are implanted into a mammal who will benefit when the protein is expressed by the cells in the mammal. In some instances, the genetically modified DCs are implanted into a mammal who will benefit when the protein is expressed and secreted by the cells in the mammal.

According to the present invention, gene constructs which comprise nucleotide sequences that encode heterologous proteins are introduced into a cell. That is, the cells are genetically altered to introduce a gene whose expression has therapeutic effect in the mammal. According to some aspects of the invention, cells from a mammal or from another mammal or from a non-human animal may be genetically altered to replace a defective gene and/or to introduce a gene whose expression has therapeutic effect in the mammal.

In all cases in which a gene construct is transfected into a cell, the heterologous gene is operably linked to regulatory sequences required to achieve expression of the gene in the cell. Such regulatory sequences include a promoter and a polyadenylation signal.

The gene construct is preferably provided as an expression vector that includes the coding sequence for a heterologous protein operably linked to essential regulatory sequences such that when the vector is transfected into the cell, the coding sequence will be expressed by the cell. The coding sequence is operably linked to the regulatory elements necessary for expression of that sequence in the cells. The nucleotide sequence that encodes the protein may be cDNA, genomic DNA, synthesized DNA or a hybrid thereof or an RNA molecule such as mRNA.

The gene construct includes the nucleotide sequence encoding the beneficial protein operably linked to the regulatory elements and may remain present in the cell as a functioning cytoplasmic molecule, a functioning episomal molecule or it may integrate into the cell's chromosomal DNA. Exogenous genetic material may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be introduced into the cell.

In addition to the gene therapy aspect of the invention, the DCs are equally useful in the context of protein-based therapy. The desired protein or therapeutic protein can be made by any means in the art. For example, a host cell transfected with a nucleic acid vector directing expression of a nucleotide sequence encoding a desired protein can be cultured in a medium under appropriate conditions to allow expression of the protein to occur. Protein can be isolated from cell culture medium, host cells, or both using techniques known in the art for purifying proteins. Once purified, partially or to homogeneity, the recombinantly produced protein or portions thereof can be utilized in compositions suitable for pharmaceutical administration as described in detail herein. The therapeutic protein can also be a synthetically derived peptide or polypeptide with the purpose of directing the immune response.

### Dosage and Formulation (Pharmaceutical Compositions)

The present invention provides a method for treating cancer. The method of the invention provides local delivery of one or more therapeutic agents to a site in need thereof. In another embodiment, the method of the invention provides delivery of one or more therapeutic agents to a proximate site of the tumor site.

The polymer platform of the invention may be administered to a patient or subject in need in a wide variety of ways. Modes of administration include intraoperatively intravenous, intravascular, intramuscular, subcutaneous, intracerebral, intraperitoneal, soft tissue injection, surgical placement, arthroscopic placement, and percutaneous insertion, e.g. direct injection, cannulation or catheterization. Any administration may be a single application of a composition of invention or multiple applications. Administrations may be to single site or to more than one site in the individual to be treated. Multiple administrations may occur essentially at the same time or separated in time.

In certain embodiments, the polymer platform of the invention is administered during surgical resection or debulking of a tumor. For example, in patients undergoing surgical treatment of a tumor, the polymer platform may be administered to the tumor site in order to further treat the tumor, prevent the growth of the tumor, or prevent the formation of additional tumors.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Administration of the therapeutic composition in accordance with the present invention may be continuous or intermittent, depending, for example, upon the recipient's physiological condition, whether the purpose of the administration is therapeutic or prophylactic, and other factors known to skilled practitioners. The administration of the compositions of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into the tumor. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

The therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The cells encompassed in this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

### Methods

The present invention provides a method for treating a disease or disorder in a subject, comprising administering a polymer platform comprising one or more releasable therapeutic agents to a treatment site in the subject.

For example, in certain embodiments, the method is used to treat or prevent cancer in a subject. In certain embodiments, the method comprises administering an effective amount of a polymer platform described herein to a subject diagnosed with cancer, suspected of having cancer, or at risk for developing cancer. In certain aspects, polymer platform is contacted to a cell or tissue where cancer is present or at risk for developing. In one embodiment, the polymer platform is administered to a tumor site. In one embodiment, the polymer platform is administered to a site within the subject where a tumor is removed. For example, in one embodiment, the method comprises the removing or debulking of all or some of a tumor, and intraoperatively administering the polymer platform at the site in which the tumor was removed or debulked. For example, in subjects undergoing surgical treatment of a tumor, the polymer platform may be administered to the tumor site in order to further treat the tumor, prevent the growth of the tumor, or prevent the formation of additional tumors. Subjects to which administration of the polymer platform of the invention is contemplated include, but are not limited to, humans and other primates, mammals including commercially relevant mammals such as non-human primates, cattle, pigs, horses, sheep, cats, and dogs.

As described elsewhere herein, the polymer platform of the invention releases one or more therapeutic agents to the treatment site. For example, in one embodiment, the administered polymer platform releases one or more anti-tumor agents to a tumor microenvironment. In one embodiment, the polymer platform releases one or more immunomodulators to the tumor microenvironment. In one embodiment, the polymer platform releases one or more protective agents to healthy tissue surrounding the tumor.

In one embodiment, the method comprises the release of a radiosensitizer to the tumor site. In certain aspects, this allows for enhanced efficacy of subsequent radiation therapy administered to the patient. In certain aspects, this allows for the use of a lower dose of radiation during subsequent radiation therapy administered to the patient. The method also includes the release of other anti-tumor sensitizing agents, including agents that sensitize the tumor to subsequent therapies, including chemotherapies or hypothermia. In one embodiment, the method comprises administering the subsequent anti-tumor therapy, including radiation, chemotherapy, and/or hypothermia, to the subject after the polymer platform was implanted and allowed to deliver its embedded therapeutic agents for a defined period.

In one embodiment, the method comprises administering a protective polymer platform to regions of healthy tissue at or near the tumor site. This protective polymer platform may restrict the delivery of anti-tumor agents to healthy tissue. In certain aspects, the protective polymer platform delivers protective agents to the healthy tissue, to help spare the healthy tissue during subsequent radiation, chemotherapy, or hypothermia treatments.

The present invention provides a method for the treatment or prevention of cancer. For example, in certain aspects, the method prevents the development of cancer, prevents the metastasis of a cancer, prevents the recurrence of a cancer, reduces the aggressiveness of a cancer, reduces the size of a cancerous tumor, and the like.

Cancers that may be treated include tumors that are not vascularized, or not yet substantially vascularized, as well as vascularized tumors. Types of cancers to be treated by the method of the invention include, but are not limited to, carcinoma, blastoma, melanoma, and sarcoma, and certain leukemia or lymphoid malignancies, benign and malignant tumors. Adult tumors/cancers and pediatric tumors/cancers are also included.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In one embodiment, the method comprises manufacture of the polymer platform of the invention. As described elsewhere herein, one or more layers of the polymer platform may be designed and constructed in standard sizes and geometries, or in certain instances may be customized for the specific patient being treated.

In one embodiment, the method comprises modifying a cell to express a therapeutic agent of interest. For example, as described elsewhere herein, in certain embodiments, the polymer platform comprises one or more genetically modified cells, modified to express a therapeutic agent.

In certain embodiments, genetically modified cell is autologous to a subject being treated. Alternatively, the cells can be allogeneic, syngeneic, or xenogeneic with respect to the subject. The genetically modified cell may be modified in vivo or ex vivo, using techniques standard in the art. For example, genetic modification of the cell may be carried out using an expression vector or using a naked isolated nucleic acid construct.

In one embodiment, the cell is obtained and modified ex vivo, using an isolated nucleic acid encoding a therapeutic agent. For example, the cell may be modified using an isolated nucleic acid encoding CCL21, or other immunomodulator. In certain embodiments, the cell is expanded ex vivo or in vitro to produce a population of cells.

The genetically modified cells may then be seeded into or onto one or more layers of polymer platform. Seeding of cells into or onto the polymer platform may be performed according to standard methods. For example, the seeding of cells onto polymeric substrates for use in tissue repair has been reported (see, e.g., Atala, A. et al., J. Urol. 148(2 Pt 2): 658-62 (1992); Atala, A., et al. J. Urol. 150 (2 Pt 2): 608-12 (1993)). Cells grown in culture may be trypsinized to separate the cells, and the separated cells may be seeded on the polymer platform. Alternatively, cells obtained from cell culture may be lifted from a culture plate as a cell layer, and the cell layer may be directly seeded onto the polymer platform without prior separation of the cells. In one embodiment, isolated cells may be dispersed within a polymer precursor solution, such that the cells become encapsulated within a formed polymer.

The seeded polymer may be incubated under standard culturing conditions, such as, for example, 37° C., 5% CO₂, for a period of time. However, it will be appreciated that the density of cells seeded within the polymer may be varied. For example, greater cell densities promote greater anti-tumor activity by the seeded cells. Selection of cell types, and seeding of cells within the polymer platform, will be routine to one of ordinary skill in the art in light of the teachings herein.

In one embodiment, the invention provides a personalized method of treatment or prevention of a disease or disorder, where the polymer platform is designed and constructed based on the disease state and characteristics of the subject being treated. For example, in one embodiment, the method comprises determining a profile of the subject based upon one or more of tumor size, tumor location, type of cancer, stage of cancer, biomarker profile of subject or tumor microenvironment, age of subject, sex of subject, family history of the subject, and the like. In one embodiment, the method comprises obtaining a sample of the tumor, for example via a biopsy, and customizing one or more layers of the polymer platform based upon the characteristics of the tumor. For example, the tumor may be classified, using standard techniques, to determine which types of therapeutic agents may be best suited to treat the tumor. In one embodiment, the method comprises designing and constructing the layers of the polymer platform to optimize polymer degradation and/or the timing and duration of therapeutic agent secretion from the polymer. In one embodiment, the polymer platform can be specifically designed with regions comprising mechanical barriers and/or protective agents corresponding to regions of the tumor microenvironment having healthy tissue. Further, the polymer platform can be specifically tuned to deliver sensitizing agents that enhance the efficacy of subsequent anti-tumor therapies, such as chemotherapy, radiation, and hypothermia.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### A Modular Polymer Platform that Delivers Cytokines and Cisplatin is Effective in Reducing Tumor Burden in an Animal Model of Head and Neck Squamous Cell Carcinoma (HNSCC)

The data presented herein demonstrates the development of a modular drug delivery device that reproducibly reduces tumor growth in vivo. In this study, a partial tumor resection model in the mouse was utilized, replicating the difficult situation observed patients in which the entire tumor is not resectable. The polymer platform comprises is a flexible sheet that is designed to be applied intraoperatively to the surgical bed after removing or debulking the tumor, and is engineered to adapt and adhere to the surgical resected tissue contours. Cisplatin has been widely used in combination with radiation as a radiosensitizer in preclinical and clinical studies (Cohen et al., 2010, Otolaryngol Head Neck Surg, 143: 109-115). It was examined whether the local delivery of cisplatin via the described polymer maximizes therapeutic index, minimizes systemic side effects, and enhances post-operative radiation treatment.

Another component to the modular platform described herein is the concept of polymer delivery of immunomodulators, which will increase the efficiency of tumor cell killing by the host's immune system. Patients with HNSCC have been well documented to exhibit local immunosuppression with depressed T-cell-mediated responses as well as depressed NK cell and antibody-dependent cell cytotoxic effects (Lim et al., 2013 Oncoimmunology 2(6):e24677; Jewett et al., 2006 Cancer Immunol Immunother 55(9):1052-63).

CCL21 (C-C motif cytokine ligand 21) is an immune active chemokine involving in immune cell chemotaxis by binding to its CCR7 receptor (Yoshida, 1998 J Biol. Chem. 273(12):7118-22). It has been previously reported that CCL21 immunotherapy mediates T cell dependent anti-tumor effect and reduces tumor burden in a lung cancer model. Both innate natural killer and specific T-cell antitumor responses are significantly increased following dendritic cell (DC)-based CCL21 therapy.

The materials and methods employed in these experiments are now described.

Polymer Fabrication

The cisplatin-releasing polymer was designed to be adequately flexible to adapt to irregular tissue contours without tearing. To meet this requirement, a wide range of mixing ratios involving two polymers: poly-ε-caprolactone (PCL) and a co-polymeric blend of poly (DL-lactide-co-ε-caprolactone) (PLCL), were evaluated in a pilot study. A 70:30 ratio of PLCL:PCL was found to offer the optimal flexibility and malleability of the PCL sheet, allowing for more facile handling by surgeons during implantation in vivo. Both PCL and PLCL were obtained from Boehringer Ingelheim and are manufactured under GMP and ISO-certified facilities, qualifying these materials' availability for future clinical testing. Polymers were dissolved in chloroform in 70:30 ratio of PLCL:PCL for 24 hours until gentle mixing (Wang, 2010, Sequential Layer Polymer Deposition [master's thesis]. Los Angeles, Calif.: UCLA Biomedical Engineering). For the cisplatin delivery, fresh cisplatin (4 mg/kg) was added to the polymer solution.

For the DC-CCL21 delivery, Fibrinogen (5 ug/ml) and Thrombin (5 IU/ml) were dissolved in PBS and 100 mM CaCL₂, respectively. A 1.5×1.5 cm² well was made of fibrin gel on top of the PCL-PLCL film for cell seeding. Cells were encapsulated in 300 μl solution of fibrinogen with or without collagen (3 mg/ml) at the ratio of 75:25. Cells were dispensed into well, followed by addition of 300 μl thrombin. After solidification, cells in the polymer were cultured in DMEM overnight until use. After spreading, the sheets were dried overnight vacuum packed, and protected from ambient light. This polymer device is designed to degrade gradually overtime to prevent extrusion and long-term giant cell foreign body response. Furthermore, material selection was limited to those that are used in FDA-approved devices. Therefore, both the intact polymer as well as its degradation products is known to be well tolerated in humans.

Mouse Model

The mice used in this study were 6-week-old C3H/HeJ mice (Jackson labs, Bar Harbor, Me.). The mice were maintained under specific pathogen-free conditions, and sterilized food and water is available ad libitum.

Animal Model Surgical Procedure

4×10⁵ cells from the well-established C3H/HeJ mouse SCCA cell line SCCVII/SF were injected into 6-week-old C3H/HeJ mice. The SCC VII/SF cell line is a spontaneously arising squamous cell carcinoma syngeneic to C3H/HeJ mice (O'Malley et al., 1997, Arch Otol, 123: 20-24). Eight mice were injected in each group unless otherwise specified. All mice were injected subcutaneously over the right flank. Tumor growth was assessed with calipers three times/week following polymer implantation for 18-31 days to evaluate the antitumor efficacy of the different treatments. Tumor growth was assessed for 18-31 days as this was the time point at which control animals required euthanization due to tumor burden. The length, width, and height (in mm) of the tumors were measured and tumor volume (mm³) was calculated according to the formula:

Tumor volume=π/6×length×width×height

When tumors reached an average size of 0.5-1 cm³, all animals underwent surgery to debulk their tumors by 50% to approximate the surgical situation when a patient's tumor is unresectable, and some tumor is left behind prior to polymer therapy. Animals were then randomly assigned to the different treatment groups. The treatment groups included: (1) no polymer; (2) plain polymer; (3) plain polymer with local cisplatin injection; (4) cisplatin polymer. For cytokine studies mice were grouped into: (1) no polymer; (2) plain polymer; (3) plain polymer with intratumoral injection of recombinant CCL21 twice a week; (4) polymer containing parental dendritic cells; (5) polymer containing dendritic cells secreting CCL21 (DC-CCL21). Each tumor bed was completely covered with the polymer according to the treatment group. The polymer was placed over each tumor and draped over the tumor edges.

Radiation Therapy

After tumor debulking (described above) animals were assigned to various treatment groups. For the RT experiments, the treatment groups were: (1) No treatment (no polymer addition, surgical debulking only); (2) No treatment (no polymer addition, surgical debulking only)+RT; (3) cisplatin polymer alone; and (4) cisplatin polymer+RT. On postsurgical day number three, the mice were anaesthetized and positioned in a Lucite jig with lead shielding the body, except for the tumor site, which was irradiated using a Gamma cell 40 irradiator (Cs-137 source; Atomic Energy of Canada Ltd., Ottawa, Canada) at a dose rate of approximately 0.6 Gy/min. Tumors were irradiated with a total dose of 16 Gy given in 4 Gy fractions on 4 consecutive days. Dosimetry was performed using Harshaw TLD-100H (LiF:Mg, Cu, P) and film (GAFCHROMIC EBT2, International Specialty Products, Wayne, N.J.) and calibrated against a clinical cobalt-60 irradiator (Theratron-1000, MDS Nordion, Ontario) indicating that leakage and scatter of gamma rays to the shielded areas reached about 10.9-14.1% of the total dose. Dose fractionation was chosen to allow for repair, which is required for radiosensitization by cisplatin. The size of dose was based primarily on the fact that these tumors grow considerably more rapidly than in the clinic and standard 2 Gy doses are inadequate to compensate for rapid cell division. In addition, doses of >2 Gy per fraction are becoming increasing popular clinically either as planned homogeneous or inhomogeneous dose distributions, so the use of 4 Gy fraction sizes is not clinically irrelevant (Stuschke et al., 2010, Frontiers of Radiation Therapy and Oncology, 42: 150-156). Cisplatin is a known radiation sensitizer and the polymer platform releasing 4 mg/kg cisplatin within the radiation field allows for the amplification of radiation dose in a localized manner (Deurloo et al., 1991, Cancer Chemother Pharacol, 27: 347-353; Ning et al., 1999, Radiother Oncol, 2: 215-223). Benefits of cisplatin as a radiation sensitizer were investigated herein, and therefore the drug was not administered systemically. The length, width and height (mm) of each tumor were measured with calipers three times a week. After the animals were sacrificed, a gross necropsy and histopathological examination of the tissues surrounding the implant site was conducted. The inflammatory response to the implanted polymer was determined based on the average number of cell types present in the surrounding tissue. Tissue responses were found to be minimal (data not shown). The histopathological examination of all tissues and tumors was performed.

Cell Culture

The DC 2.4 cell line was obtained. DC 2.4 cells were isolated from mouse bone marrow and immortalized by transfection with myc and raf. DC 2.4 cells were maintained in Dulbecco's Modified Eagle's Medium (DMEM, Gibco, CA) supplied with 10% Fetal Bovine Serum and 100 units/ml Penicillin-Streptomycin. Mouse syngeneic SCCA cell SCCVII/SF were maintained in RPMI medium with 10% FBS.

Preparation of Retrovirus and CCL21 Transduction

Human CCL21 overexpressing DC2.4 cells were generated as follows. Wild-type CCL21 cDNA was cloned from human lymphoma tissue and subcloned into the retrovirus vector pLNCX (Clontech), which was cut with HindIII and HpaI. The vector contains the CMV promoter for controlling transcription of the cDNA insert and Neomycin (Fisher) resistance gene for selection. For virus production, 70 percent confluent 293T cells were cotransfected with pLNCX-CCL21 and Amphotropic (Packaging plasmid) or PLNCX and Amphotropic respectively using the Calcium Phosphate Transfection Kit (Invitrogen). Tumor cells were then transduced with high-titer producing CCL21 and pLHCX virus. Following transduction, DC 2.4-CCL21 cells were selected by G418 (200 ug/ml) and checked by ELISA for CCL21 expression.

ELISA

CCL21 concentrations in 1 cc of culture medium were determined by ELISA as recommended by the manufacturer (R&D Systems, MN). Briefly, 96-well Costar (Cambridge, Mass.) plates were coated overnight with 4 μg/ml of the appropriate anti-mouse CCL21 mAb. The wells of the plate were blocked with 10% fetal bovine serum (Gemini Bioproducts) in PBS for 1 hr. Plates were then incubated with 100 μl medium or standard buffer for 2 hours. The plate was then incubated with 2 μg/ml biotinylated mAb to the appropriate cytokine (PharMingen, San Diego, Calif.) for 2 hr, and excess Ab was washed off with PBS-Tween. The plates were incubated with avidin peroxidase, and after incubation in OPD substrate to the desired extinction, the subsequent change in color was read at 450 nm with a Microplate Reader (Molecular Dynamics, Sunnyvale, Calif.). The recombinant mouse CCL21 used as standards in the assay were obtained from the same manufacturer.

Flow Cytometry

Tumors were harvested, cut into small pieces in RPMI 1640, and passed through a sieve (Bellco Glass, Vineland, N.J.). Tumor leukocytes were isolated by digesting tumor tissue in collagenase IV (Sigma) in RPMI 1640 for 30 min with stirring at 37° C. A 10-ml syringe with a blunt-ended 16-gauge needle was used to break down the tissue further. The cell suspension was strained through a disposable plastic strainer (Fisher, Pittsburgh, Pa.) to separate free lymphocytes from tissue matrix. The cells were pelleted at 2,000 rpm for 10 min and cell pellets washed twice to remove collagenase. Leukocytes were additionally purified using a discontinuous Percoll (Sigma) gradient, collecting at the 35-60% interface after centrifugation at 1,500 rpm for 20 min at 4° C. without brake. The collected cells were washed twice in PBS and stained for flow-cytometric evaluation. After Percoll purification, the percentage of leukocytes in the cell population was approximately >95%. For staining, two or three fluorochromes (phycoerythrin, FITC, and PerCP; PharMingen) were used to gate on the CD4, and CD8 T-lymphocyte population or CD11c⁺ DCs in single-cell suspensions from tumor nodule. For T-regulatory cell quantification, T cells were doubly stained for CD4 and CD25 cell surface markers. Flow cytometric analyses were performed on a FACScan flow cytometer (Becton Dickinson, San Jose, Calif.) in the University of California Los Angeles Jonsson Cancer Center Flow Cytometry Core Facility. Gated events (10,000) were collected and analyzed using Cell Quest software (Becton Dickinson).

Statistics

Tumor growth curves were compared between treatment arms using repeated measures analysis of variance (ANOVA) models. These models contained terms for time, treatment effects and the interaction between time and treatment terms. For tumor growth experiments in which there was a significant treatment by time interaction effects (demonstrating differences in tumor growth rates between groups), the mean tumor sizes between treatment arms of interest at specific time points were compared using Bonferroni corrected two-sample t-tests. Tumor weight was compared between groups with a two-way ANOVA model containing terms for cisplatin polymer and radiation. In both types of tumor models the log transformation was used to reduce the influence of outliers. P<0.05 was considered significant.

The results of the experiments are now described.

### The Modular Polymer Device is Safe and Biocompatible in the Animal Model and is Facile for Surgical Use.

Although the tissue biocompatibility of the backbone of the polymers is well documented in the literature (Gopferich, 1996, Biomaterials, 17: 103), the safety and biocompatibility of the Chemotherapeutic Layer of the device was tested in a mouse model. In order to accelerate clinical translation, materials were selected that are currently used in medical devices with known characteristics in terms of toxicity, hypersensitivity, mutagenesis, and inflammatory responses.

The partial tumor resection model in the mouse was used. After implantation, the mice were observed daily for overt toxicity. The animals tolerated the implant well and were not observed to pick or scratch at the polymer. No signs of bleeding, swelling, or infection were noted at the incision site. When the animals were sacrificed, a histopathological examination of the tissues surrounding the implant site was conducted. The inflammatory response to the implanted polymer was found to be minimal.

### Cisplatin Secreting Polymer Reduces Tumor Burden in Head and Neck Cancer

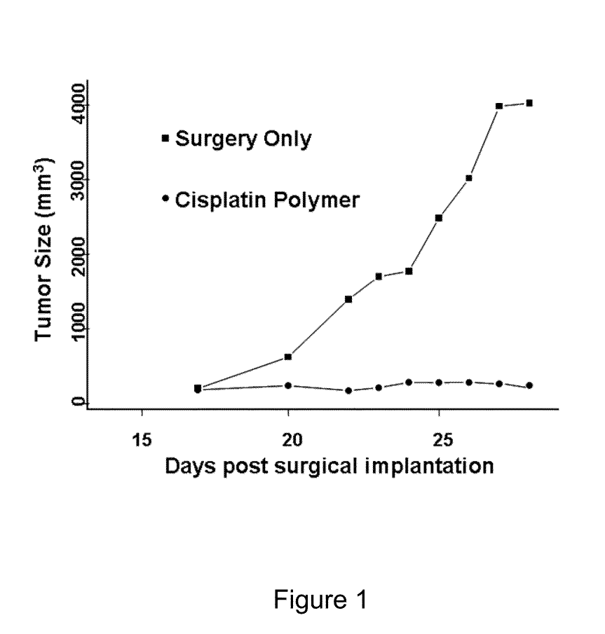

The antitumor efficacy of the chemotherapeutic layer of the polymer platform was evaluated in murine models of head and neck cancer. The results presented herein using this novel polymer platform demonstrate a significant reduction in tumor growth. The cisplatin secreting polymer effectively reduced SCCVII/SF tumors in the C3H/HeJ mice by over 10-fold on day 25 (P<0.01) (FIG. 1) as compared to control (surgical debulking only), plain polymer, and plain polymer+intratumoral cisplatin injection groups (P<0.01). This decrease in tumor burden was confirmed by excising the tumors that had been treated with the cisplatin polymer (FIG. 1). The data shows that the cisplatin-secreting polymer is more effective against head and neck cancer than the plain polymer plus cisplatin given as an intratumoral bolus injection.

Cisplatin Secreting Polymer Enhances the Efficacy of Radiation Therapy Mice treated with radiation, with or without cisplatin polymer implantation, demonstrated significant reduction in tumor regrowth compared to the control group (radiation alone, 22% the size of control, P=0.005; radiation with cisplatin polymer 12% the size of control, P=0.003). In these experiments tumors were irradiated with a total dose of 16 Gy given in 4 Gy fractions on 4 consecutive days.

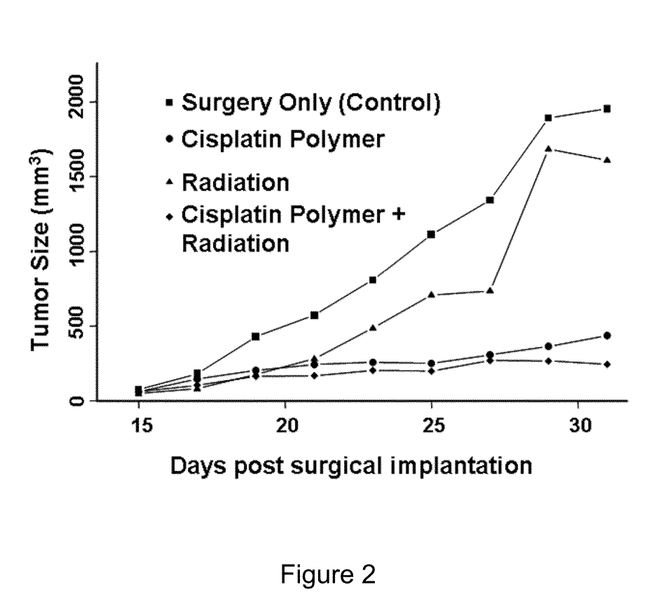

A closer comparison between the two different radiation treatment groups revealed lower tumor burden in the group also implanted with cisplatin polymer (FIG. 2) (53% the size of the radiation alone group). An overall analysis of the four treatment arms found that both cisplatin polymer and radiation resulted in significantly reduced tumor size over time (p=0.03 and 0.001, respectively). This observation was corroborated by the statistically significant lower tumor weight noted among mice treated with cisplatin polymer and concomitant radiation compared to the radiation alone group and the control group (FIG. 2) (32% the weight of the radiation alone group, P=0.05; 9% the weight of the control group treated with debulking alone, P=0.0002).

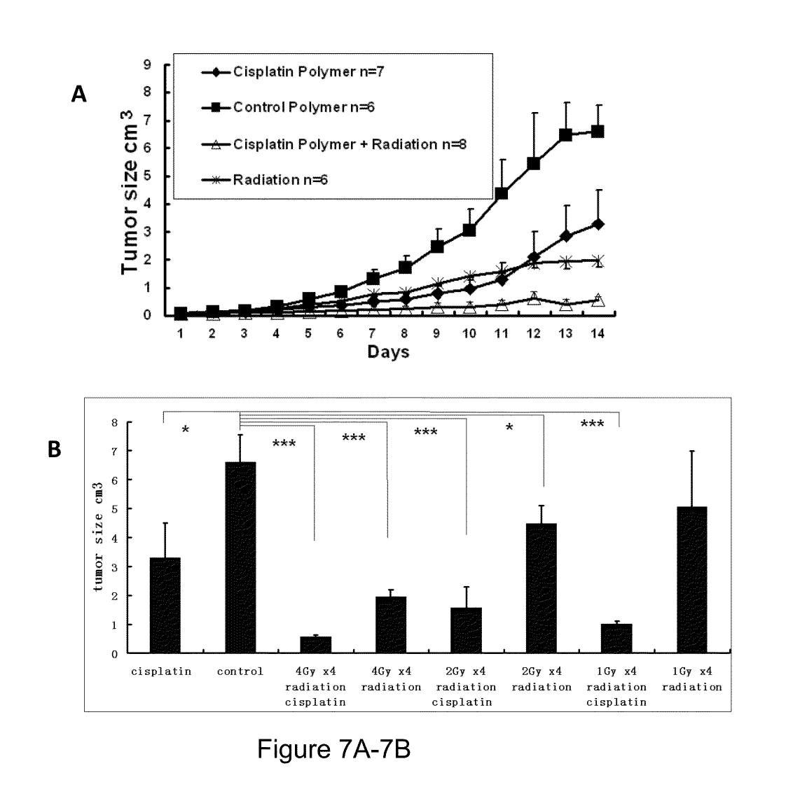

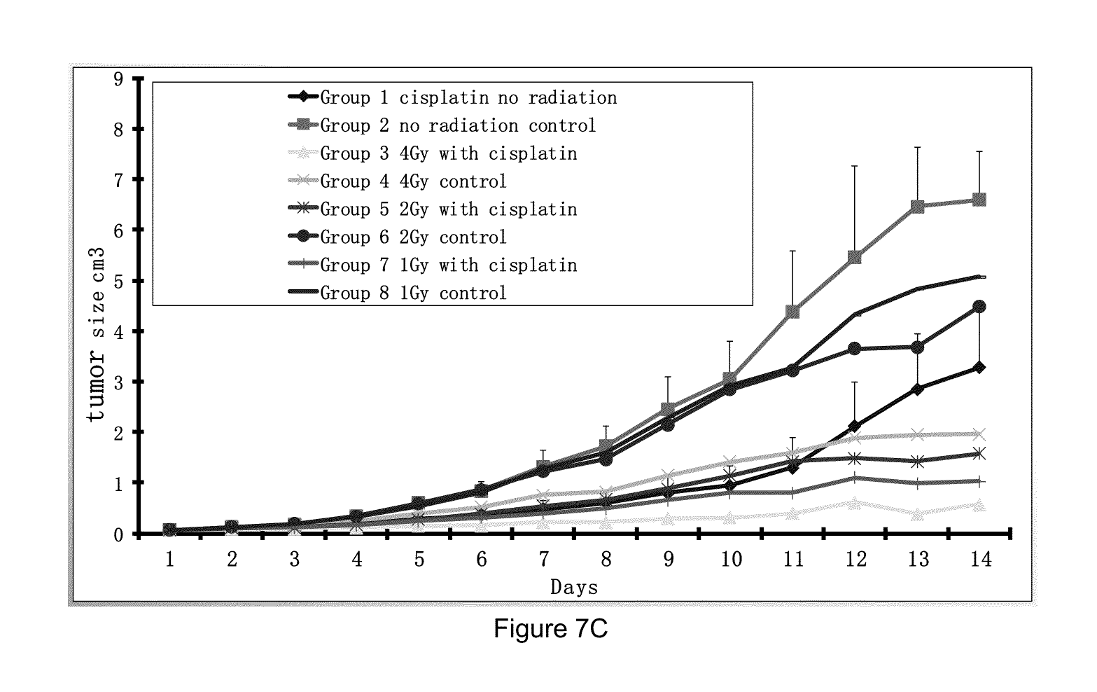

Further, it is demonstrated herein that tumor size is decreased in cisplatin polymer compared to a control polymer (FIG. 7A). Additional experiments were conducted to examine whether the effects of the cisplatin polymer in enhancing radiation therapy is seen over various doses of radiation. It is demonstrated that the cisplatin polymer enhanced the efficacy of radiation therapy at 1 Gy, 2 Gy, and 4 Gy doses (FIG. 7B and FIG. 7C); the doses were repeated over 4 days. It is noted that the 4 Gy×4 dose regime in a mouse is equivalent to 70 Gy (full does radiation therapy) in a human. This demonstrates that cisplatin polymer allows for a lower dose of radiation to be administered, thereby reducing the potential for adverse side effects.

These results are quite promising that the polymer platform can direct the highest dose of radiation therapy to the tumor and spare surrounding normal tissues.

Dendritic Cells Overexpressing CCL21 (DC-CCL21) can Grow and Successfully Secrete CCL21 from the Polymer.

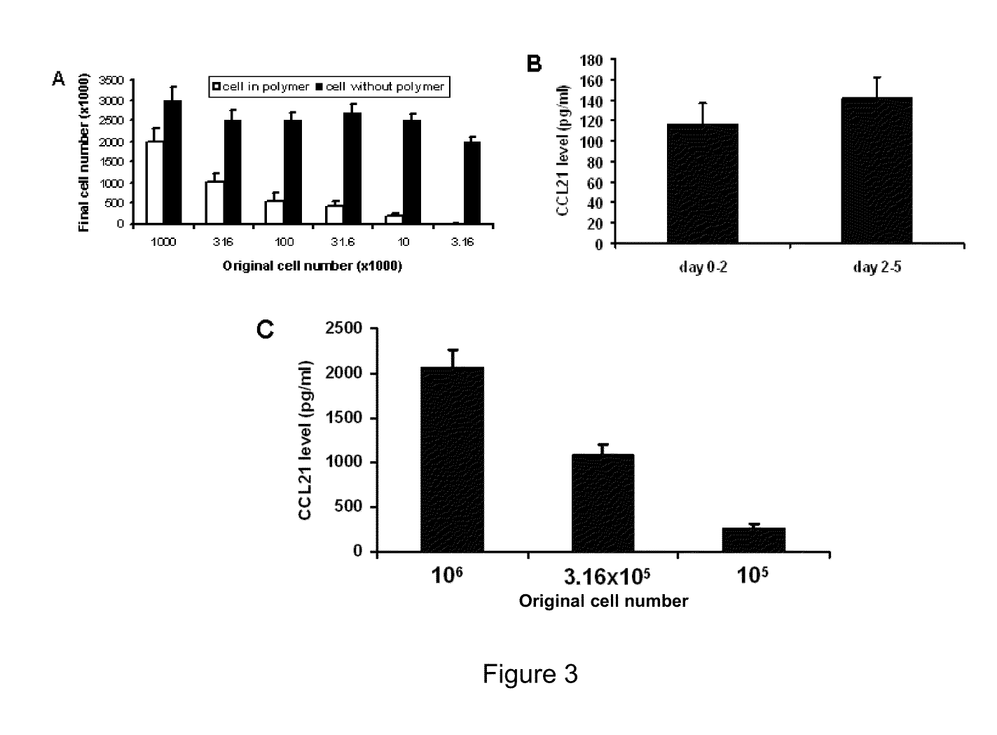

To investigate the biocompatibility of the polymer in culturing dendritic cells in vitro, the capacity of dendritic cells (DC) to grow and survive in vitro while in the polymer was investigated. Cells were plated at different initial densities within the polymer or directly onto plates (without the polymer) in vitro. Five days later, dendritic cells in the 10⁶/well group grew to 2×10⁶ in the polymer, or to 3×10⁶ without polymer (FIG. 3A). Although a less robust growth rate in the DC-polymer group was noted, DC grown in the polymer exhibited similar morphology as those cultured without polymer.

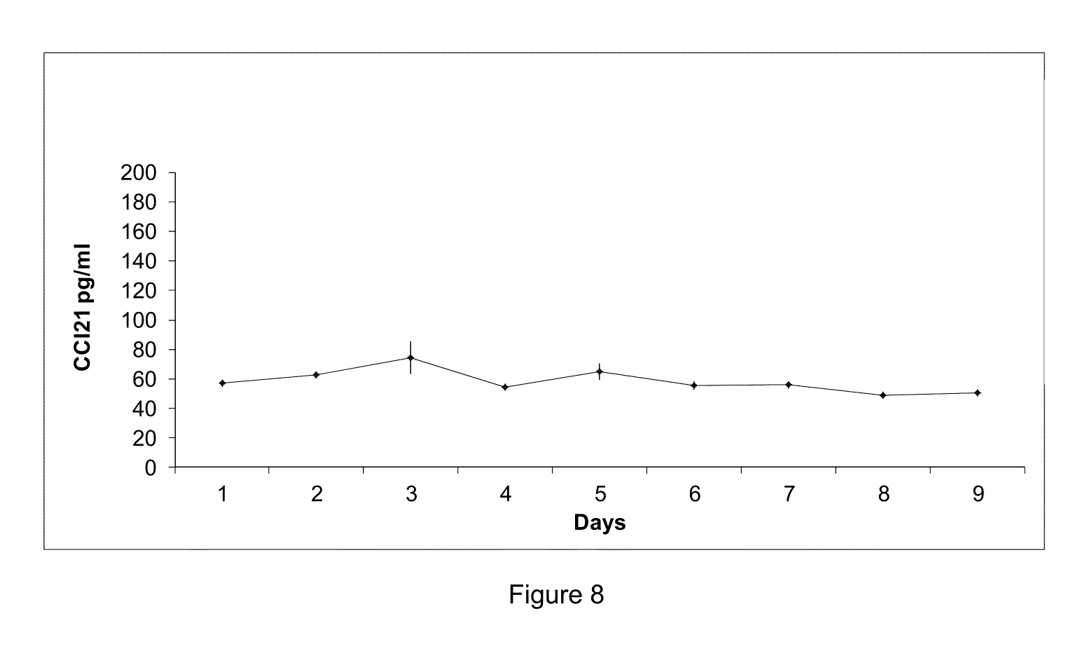

To investigate the efficacy of CCL21 production from polymer-cultured dendritic cells (DC-CCL21), ELISA was performed on medium collected from the in vitro plates after 5 days of culture. Time dependent experiments demonstrated continuous release of CCL21 from 10⁵ dendritic cells (FIG. 3B). Further, FIG. 8 demonstrates that CCL21 release was similar over 9 days. This data revealed a reproducible correlation between initial cell density and final CCL21 production (FIG. 3C). The maximum yield of CCL21 was 2058±203 pg/ml from 10⁶ cells over 5 days, and this is therefore the dose that was used in all subsequent experiments.

### DC-CCL21 Secreting Polymer Reduces Tumor Burden in Head and Neck Cancer

The antitumor efficacy of the DC-CCL21 polymer platform was evaluated in murine models of head and neck cancer. The results using this novel polymer platform demonstrate a significant reduction in tumor growth. Animals were randomly assigned to the following five treatment groups: (1) no polymer; (2) plain polymer; (3) plain polymer with intratumoral injection of recombinant CCL21 twice a week; (4) polymer containing parental dendritic cells; (5) polymer containing dendritic cells secreting CCL21 (DC-CCL21).

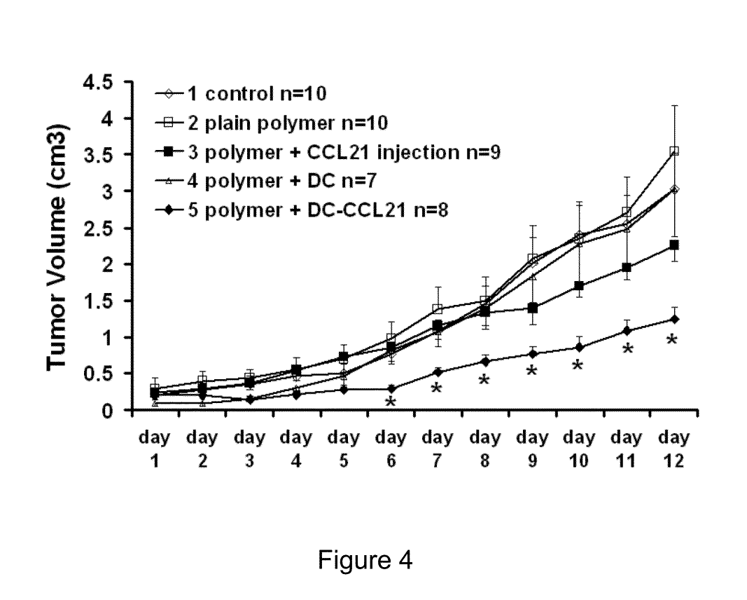

The DC-CCL21 secreting polymer effectively reduced SCCVII/SF tumors in the C3H/HeJ mice by 41% as compared to control groups (p<0.01) (FIG. 4). Plain polymer or polymer+DC treatment showed no significant difference in tumor volume as compared to the control group (p>0.05). This data shows that the CCL21-secreting polymer is more effective against head and neck cancer than the plain polymer plus CCL21 given as an intratumoral bolus injection twice weekly.

### DC-CCL21 Recruits DC, T Cells and Inhibits Treg Cells to the Tumor Site.

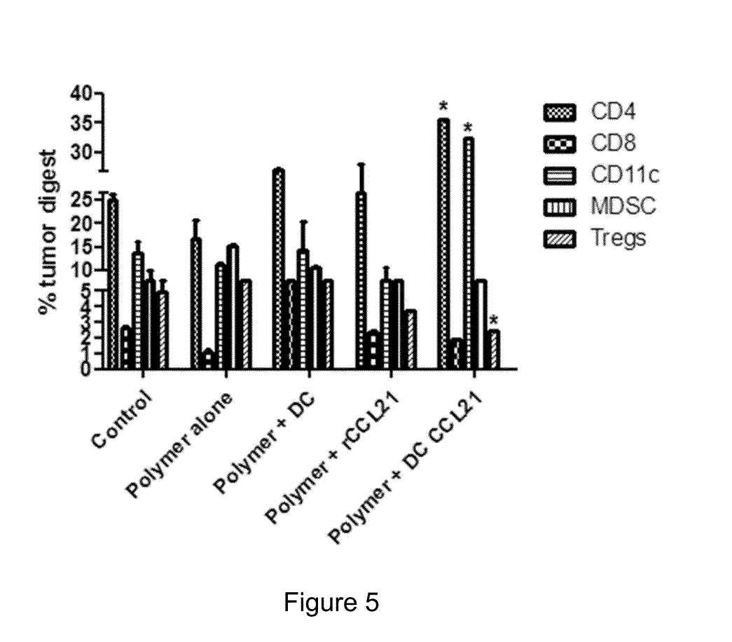

Animals treated with DC-CCL21 polymer exhibited a significant increase in the frequency of CD4+ T cell and CD 11c+ dendritic cells, as well as a marked decrease in CD4+ CD25+ regulatory T cells infiltrating the tumor sites (FIG. 5). These data suggest the antitumor effect of polymer-based DC-CCL21 treatment may, at least partially, be due to enhanced immune activity around tumor site.

### DC-CCL21 Polymer Inhibits Epithelial-to-Mesenchymal Transition (EMT) in HNSCC Tumors.

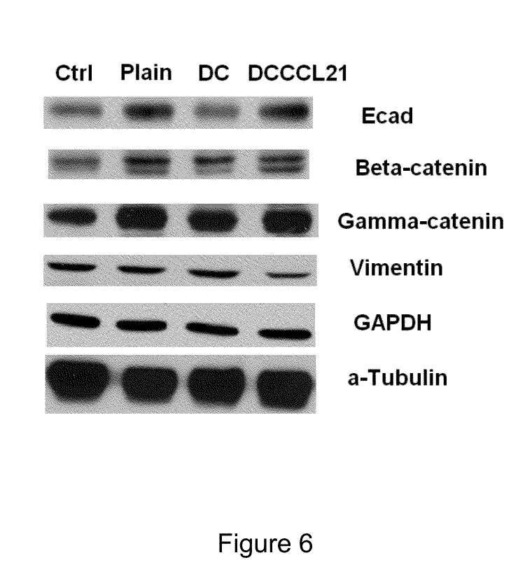

The effect of DC-CCL21 polymer on epithelial-to-mesenchymal transition (EMT), a potential mechanism of metastasis, was investigated. Tumor samples were harvested 12 days after implantation and were analyzed by immunoblotting against EMT markers. Epithelial markers, including E-cadherin, beta-catenin and gamma-catenin, were increased, whereas the mesenchymal marker vimentin was decreased in the DC-CCL21 polymer treated tumors (FIG. 6). These data suggest that DC-CCL21 polymer treatment may lead to a less mesenchymal status that attenuates tumor invasion, and by promoting an epithelial phenotype, can also sensitize tumors to other forms of therapy.

### Concomitant CCL21 and Cisplatin Secreting Polymer Further Reduced Tumor Burden

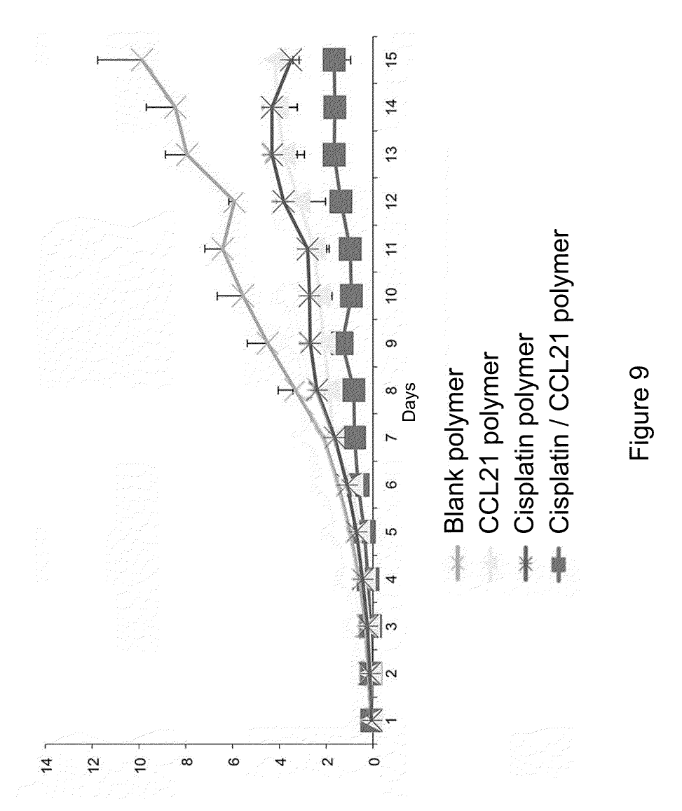

It was investigated whether polymer comprising cisplatin and CCL21 further reduced tumor burden compared to the polymers releasing only one of cisplatin or CCL21. It was observed that polymer releasing both cisplatin and CCL21 resulted in reduced tumor burden than control polymer, polymer releasing CCL21 alone, and polymer releasing cisplatin alone (FIG. 9). Preferably, the polymers were bi-layered polymers. In some instances, the bilayered polymers comprised genetically modified DCs secreting CCL21 and released cisplatin whereas other instances the bilayered polymers released CCL21 protein and cisplatin. The polymers examined have directionality, in that they secrete only in the direction of the tumor.

### Modular Drug Delivery Device

Presented herein is the development of a modular drug delivery device that reproducibly reduces tumor growth and enhances the efficacy of RT in vivo after partial tumor resection. The experiments presented herein use a partial tumor resection model in the mouse, replicating the difficult situation seen in patients in which the entire tumor is not resectable. The device is a flexible sheet that is designed to be applied intraoperatively to the surgical bed after removing or debulking the tumor, and is engineered to adapt and adhere to the surgical resected tissue contours.

The combined use of radiotherapy and chemotherapy has been effective in improving the therapeutic index of radiation therapy for a variety of human cancers (Ning et al., 1999, Radiother Oncol, 2: 215-223). Cisplatin is a highly effective anticancer agent and has been widely used in combination with radiation as a radiosensitizer in preclinical and clinical studies (Ning et al., 1999, Radiother Oncol, 2: 215-223; Gopferich et al., 1996, Biomaterials, 17: 103; Yu et al., 1988, NCl Monogr, 6: 137-140). The successful use of chemotherapeutic agents as radiosensitizers is dependent on enhanced tumor cell killing without increased normal tissue toxicity. The local delivery of synergistic agents is expected to maximize therapeutic index, minimize systemic side effects, and enhance post-operative radiation treatment. The data presented herein shows that the cisplatin-secreting polymer is more effective against head and neck cancer than the plain polymer plus cisplatin given as an intratumoral bolus injection. This enhanced antitumor activity is likely due to a more durable sustained release of cisplatin from the polymer platform increasing the interaction time with the tumor cells.

Some advantages of this polymer system over conventional brachytherapy are: better control of dose distribution, elimination of radioprotection and safety issues for the patient and the patient's family, and treating personnel. An important psychological factor is that the patient's daily activities are not restricted during the entire treatment time. An additional benefit of this polymer system includes prophylaxis against tumor recurrence following resection. Viable squamous cell carcinoma (SCC) cells have been recovered from the surgical wound following neck dissection and were shown to be capable of growing as colonies in vitro; theoretically, these may implant and cause cancer recurrence (Vicram and Misra, 1994, Head Neck, 16: 155-157). Therefore, exposing such cells to this polymer system in combination with external beam radiation therapy (EBRT), before they implant (while they are isolated and fragile), may decrease the chance of implantation.

Another attractive addition to the modular platform is the concept of polymer delivery of immunomodulators, which increases the efficiency of tumor cell killing by the host's immune system. Patients with HNSCC have been well documented to exhibit local immunosuppression with depressed T-cell-mediated responses as well as depressed NK cell and antibody-dependent cell cytotoxic effects (Jewett et al., 2006, 55: 1052-1063). Gene therapy approaches include replacement gene therapy, suicide gene therapy, and immunotherapy. Despite these many approaches, gene therapy still has been limited significantly (Xian et al., 2005, Arch Otolaryngol Head Neck Surg, 131: 1079-1085). The role of cytokines in tumor regression is now well established (Jewett et al., 2006, 55: 1052-1063). The major limitation for the clinical use of cytokines is the lack of a simple and effective protocol for the local and sustained delivery of cytokines to the tumor milieu.

Prior reports have reported that secretion of CCL21 by HNSCC cells and by other paracrine sources can combine to promote activation of CCR7 prosurvival signaling associated with tumor progression (Yoshida et al., 1998 J. Biol. Chem. 273 (12): 7118-22; Sharma et al., 2000 J. Immunol. 164(9):4558-6). Without wishing to be bound by any particular theory, the seeming disparity in the data may be explained by physiologic versus pharmacologic doses of CCL21. The present efforts to produce effective cancer therapy focus on methods to address the deficits in the tumor microenvironment. To restore tumor antigen presentation and antitumor effector activities CCL21 (secondary lymphoid chemokine, SLC) is utilized, which is known to recruit DC, T, NK and NKT cells (Sharma et al., 2000 J. Immunol. 164(9):4558-63; Yang et al., 2006 Cancer Res. 66(6):3205-13; Vaquette et al., 2006 Biomed Mater Eng 16(4 Suppl):S131-6; Shaikh et al., 2008 Cells Tissues Organs 188(4):333-46). The recruitment of NK and NKT cells is advantageous because these effectors can recognize tumor targets in the absence of MHC expression (Vaquette et al., 2006 Biomed Mater Eng 16(4 Suppl):S131-6; Shaikh et al., 2008 Cells Tissues Organs 188(4):333-46). The capacity of CCL21 to attract DCs is a property shared with other cytokines (Wu et al., 2008 Immunobiology 213(5):417-26). However, CCL21 may be distinctly advantageous because of its capacity to elicit a type I cytokine response in vivo (Loeffler et al., 2009 Cancer Immunol Immunother 58(5):769-75). It has been demonstrated previously that CCL21 administered intratumorally elicits potent antitumor responses in murine cancer models (Wu et al., 2008 Immunobiology 213(5):417-26; Loeffler et al., 2009 Cancer Immunol Immunother 58(5):769-75; Bogunovic et al., 2009 Proc Natl Acad Sci USA 106(48):20429-34; Flavell et al., 2010 Nat Rev Immunol 10(8):554-67; Sharma et al., 2000 J. Immunol. 164(9):4558-63. Vicari et al (Schuler et al., 2003 Curr Opin Immunol. 15(2):138-47) substantiated the findings in a colon cancer model. They demonstrated that CCL-21 transduced colon cancer cells had reduced tumorigenicity that was attributed to both immunological and angiostatic mechanisms (Schuler et al., 2003 Curr Opin Immunol. 15(2):138-47). Arenberg et al. (2001 Cancer Immunol Immunother (11):587-92) have also reported that CCL21 inhibits human lung cancer growth and angiogenesis in a mouse model. In addition to its immunotherapeutic potential, CCL21 has been found to mediate potent angiostatic effects (Flavell et al., 2010 Nat Rev Immunol 10(8):554-67, Sharma et al., 2000 J. Immunol. 164(9):4558-63), thus adding additional support for its use in cancer therapy.

As presented herein, the efficacy of CCL21 in the immunomodulator layer of the polymer platform was studied. Herein, it is demonstrated that DC are able to survive and secrete functional CCL21 when grown in the polymer. The DC-CCL21 secreting polymer significantly reduces tumor burden; recruits DC and T cells while inhibiting Treg cells at the tumor site; and effectively inhibits epithelial-to-mesenchymal transition (EMT) in HNSCC tumors.

Collectively, the experiments presented herein demonstrate the design and synthesis of a biocompatible modular polymer platform that improves the outcome for patients with advanced or recurrent OSCC. The surgical demand in such a setting is for wider resection or, in some instances when the tumor is fixed to the underlying vital structures, to debulk large tumors. Unfortunately, local failure in these cases is at least 40% or greater. The polymer wrap is biocompatible; is slowly degradable; and can serve as a platform to deliver immunomodulators and chemotherapeutic agents so as to most effectively kill tumor cells in the proximity of the polymer application. This polymer wrap is designed to be applied intraoperatively to the surgical bed after removing or debulking the tumor, thus allowing for enhanced post-operative radiation treatment, and also functioning as a platform for the delivery of immunomodulators. The data presented herein demonstrates that the polymer system is well tolerated and that CCL21, and cisplatin therapy combined with RT generates a potent antitumor immune response against HNSCC.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A modular polymer platform comprising two or more therapeutic agents releasable from a polymeric substrate, wherein at least one of the two or more therapeutic agents is an anti-tumor agent.

2. The modular polymer platform of claim 1, wherein at least one of the two or more therapeutic agents is an immunomodulator.

3. The modular polymer platform of claim 2 wherein the immunomodulator is selected from the group consisting of CCL21, IL-2, IL-6, IL-8, IL-7, IL-10, IL-12, interferon, G-CSF, imiquimod, CCL3, CCL26, CXCL7, oligodeoxynucleotides, and glucan.

4. The modular polymer platform of claim 2, wherein the substrate comprises a cell genetically modified to express the immunomodulator.

5. The modular polymer platform of claim 1, wherein the anti-tumor agent is a chemotherapeutic agent.

6. The modular polymer platform of claim 1, wherein the polymeric substrate comprises two or more layers, and wherein each layer has associated therewith at least one therapeutic agent.

7. The modular polymer platform of claim 6, wherein a first layer is a hydrogel.

8. The modular polymer platform of claim 6, wherein a second layer is a polymer matrix.

9. The modular polymer platform of claim 8, wherein the polymer matrix comprises PCL and PLCL.

10. The modular polymer platform of claim 6, wherein the first layer and second layer release the at least one therapeutic agent comprised therein at different rates.

11. The modular polymer platform of claim 1, wherein polymeric substrate comprises an impermeable backing layer.

12. The modular polymer platform of claim 1, wherein at least one of the two or more therapeutic agents is a radiosensitzer.

13. The modular polymer platform of claim 1, wherein at least one of the two or more therapeutic agents is a radioprotective agent.

14. A method of treating cancer, the method comprising contacting tissue at or near the site of a tumor in a subject with a polymeric substrate that releases two or more therapeutic agents, wherein at least one of the two or more therapeutic agents is an anti-tumor agent.

15. The method of claim 14, wherein at least one of the two or more therapeutic agents is an immunomodulator.

16. The method of claim 15, wherein the immunomodulator is selected from the group consisting of CCL21, IL-2, IL-6, IL-8, IL-7, IL-10, IL-12, interferon, G-CSF, imiquimod, CCL3, CCL26, CXCL7, oligodeoxynucleotides, and glucan.

17. The method of claim 15, wherein the substrate comprises a cell genetically modified to express the immunomodulator.

18. The method of claim 15, wherein the anti-tumor agent is a chemotherapeutic agent.

19. The method of claim 15, wherein the polymeric substrate comprises two or more layers, and wherein each layer has associated therewith at least one therapeutic agent.

20. The method of claim 19, wherein a first layer of the substrate is a hydrogel.

21. The method of claim 19, wherein a second layer of the substrate is a polymer matrix.

22. The method of claim 21, wherein the polymer matrix comprises PCL and PLCL.

23. The method of claim 19, comprising releasing the at least one therapeutic agent comprised within the first and second layer at different rates.

24. The method of claim 14, wherein the cancer is head and neck squamous cell carcinoma (HNSCC).

25. The method of claim 14, wherein the substrate is administered to the subject during surgical resection of at least part of the tumor.

26. The method of claim 14, further comprising administering a low dose of radiation therapy to the subject.

27. A method of claim 14, further comprising profiling the cancer of a subject and designing the polymeric substrate based upon the profiling of the cancer.

28. The method of claim 27, wherein the profiling comprises obtaining a sample of the cancer and determining the drug sensitivity of the cancer.

29. The method of claim 27, wherein the profiling comprises obtaining a biomarker profile of the subject.

30. The method of claim 27, wherein the design of the polymer platform comprises determining at least one of the identities, concentration, and release characteristics, of each of the two or more therapeutic agents of the platform.
